(19) United States
(12) Patent Application Publication
(10) Pub. No.: US 2025/0160333 A1
(43) Pub. Date: May 22, 2025

(54) CONTROL OF PLANT PESTS BY MICROBIAL AGENTS

(71) Applicant: ACIES BIO D.O.O., Ljubljana (SI)

(72) Inventors: Tina KOGEJ, Ljubljana (SI); Gregor KOSEC, Ljubljana (SI); Olivier ETCHEPARE, Ljubljana (SI); Stefan FUJS, Ljubljana (SI)

(21) Appl. No.: 18/832,472

(22) PCT Filed: Jan. 27, 2023
(86) PCT No.: PCT/EP2023/052092

§ 371 Date: Jul. 23, 2024

(30) Foreign Application Priority Data
Jan. 28, 2022 (EP) 22153943.0

(51) Int. Cl. A01N 63/20; A01N 25/04; A01N 25/30; A01P 1/00; C12N 1/20; C12R 1/00

(52) U.S. Cl. CPC A01N 63/20; A01N 25/04; A01N 25/30; A01P 1/00; C12N 1/20; C12R 2001/00

(57) ABSTRACT

The present invention relates to means and methods comprising Paraburkholderia bacteria for use in preventing and/or controlling plant pests, in particular Erwinia amylovora which constitutes the causative agent of fire blight. The present invention thus relates to novel non-medical uses of Paraburkholderia bacteria for preventing and/or controlling pests on a plant and/or on a temporary part of a plant and/or being located inside of a plant. Furthermore, the present invention relates to methods and compositions comprising Paraburkholderia bacteria for preventing and/or controlling such pests on a plant and/or on a temporary part of a plant and/or being located inside of a plant as well as to the use of Paraburkholderia bacteria for the preparation of such compositions. Further provided are kits comprising components used in the methods and compositions of the invention. The inventive use of Paraburkholderia bacteria for the prevention and/or control of plant pests are, without being limiting, particularly useful in commercial crop protection campaigns, particularly against the causative agent of fire blight. Erwinia amylovora.

The present invention relates to means and methods comprising Paraburkholderia bacteria for use in preventing and/or controlling plant pests, in particular Erwinia amylovora which constitutes the causative agent of fire blight. The present invention thus relates to novel non-medical uses of Paraburkholderia bacteria for preventing and/or controlling pests on a plant and/or on a temporary part of a plant and/or being located inside of a plant. Furthermore, the present invention relates to methods and compositions comprising Paraburkholderia bacteria for preventing and/or controlling such pests on a plant and/or on a temporary part of a plant and/or being located inside of a plant as well as to the use of Paraburkholderia bacteria for the preparation of such compositions. Further provided are kits comprising components used in the methods and compositions of the invention. The inventive use of Paraburkholderia bacteria for the prevention and/or control of plant pests are, without being limiting, particularly useful in commercial crop protection campaigns, particularly against the causative agent of fire blight, Erwinia amylovora.

A novel use of Paraburkholderia bacteria to prevent and/or control pests on a plant and/or on a temporary part of a plant and/or being located inside of a plant is provided. The bacteria can be delivered in agricultural formulations and can be applied to crops to achieve prevention or control of pest infection, in particular, pest infection by the causing agent of fire blight, Erwinia amylovora.

Fire blight caused by the bacterium Erwinia amylovora is a common and severe disease that affects a wide range of plants including plants of the family Rosaceae which contains many economically important crop plants, including inter alia apples and pears. Fire blight can greatly reduce crop yield and marketability in the current season by infecting plants. A single severe outbreak of the disease can disrupt orchard production for several years (Zhao et al, 2019). Fire blight disease is indigenous to North America and has spread to more than 50 countries around the world, including North America, Central America, Europe, North Africa, the Middle East, Oceania, and Asia (EPPO 2020).

Fire blight occurs in almost all pome fruit production regions of the world and causes damage of economic importance in extremely susceptible plant species which may inter alia include pear (Pyrus species) and quince (Cydonia sp.). Apple, crabapple (Malus sp.), and firethorns (Pyracantha sp.) may also be frequently damaged. Fire blight may also infect hawthorn (Crataegus sp.), Spiraea, Cotoneaster, toyon (Photinia sp.), juneberry or serviceberry (Amelanchier sp.), loquat (Eriobotria), mountain ash (Sorbus sp.), and other related plants. The disease can destroy crop trees or ornamental shrubs. Fire blight affects all above-ground plant organs, causing their dieback. In spring, canker can appear in infected trees as soon as the active growth begins. Cankers are small to large areas of dead bark on branches, twigs, or trunks of infected trees killed by the pathogen during previous seasons. The first sign of infection is a bacterial ooze exuding from cankers which can provide a source of inoculum that can be transmitted to other host plants, mainly by spreading from active lesions. The most common and characteristic symptoms are wilt and death of flower clusters, withering and death of shoots and twigs, and blight of leaf, fruit, nimb and trunk (EPPO 2020). The infection on pear trees is visible as infected flowers and flower stems wilt and turn black and/or young fruit shrivel and blacken.

There are no known curative agents that can remove E. amylovora from infected plants (EFSA Panel on Plant Health (PLH), 2014). Control of fire blight is therefore mainly based on prophylactic measures which include elimination of inoculum reservoirs, particularly crop debris, weeds, and the use of certified seedlings as pathogen-free planting material. The pruning of diseased twigs and branches in combination with copper-based formulation or antibiotics are the common strategy for managing fire blight disease. Crop surveillance and monitoring are necessary, as well as certification programs, to ensure the sanitary quality of the plants. Other approaches aim to modify the susceptibility of plants to the pathogen, for example by elicitation of the plant's natural defences (Wöhner et al., 2017; EPPO 2020).

Chemical control methods have not advanced significantly in the last 50 years. There are a limited number of products available, generally with moderate efficacy, such as copper compounds and certain antibiotics that are applied preventatively (EFSA Panel on Plant Health, 2014). Historically, the most significant use of antibiotics on plants worldwide has been for control of fire blight of apple and pear. Streptomycin has been used for control of fire blight in the USA since its introduction for plant use in 1955. In Europe, streptomycin is either not permitted, used on an emergency basis, or is used regularly, depending on the country. Besides streptomycin, oxytetracycline is registered on pear for control of E. amylovora. Gentamicin has been used to control fire blight of apple and pear in Mexico. In Israel, oxolinic acid is used to manage fire blight of apple, pear, and related plants, especially in areas where E. amylovora has become resistant to streptomycin. Because of its relatively high efficacy and low phytotoxicity, streptomycin has been the antibiotic of choice in most regions (McManus et al, 2002).

Biological control of fire blight using beneficial microorganisms offers a powerful and eco-friendly alternative as well as a complementary approach to substitute the frequent use of synthetic pesticides. Antagonistic microorganisms are a desirable alternative to antibiotics and copper treatments if their efficacy is comparable to that of the most effective controls regardless of the environmental conditions. Therefore, many studies have been focusing on the identification of potential biological control agents of fire blight, such as antagonistic microorganisms (Mikiciński et al., 2016; Ait Bahadoua et al., 2018).

Several biological control agents-based products have been developed and marketed worldwide for control of fire blight, and others are in the process of registration. Bacterial microflora antagonistic to E. amylovora have been isolated and identified from the natural habitat of the pathogen (healthy and diseased plants, particularly stigmas) and from soil. Biological control agents with in vitro antagonistic activity towards E. amylovora include Gram-negative bacteria Pantoea agglomerans C9-1 (syn. P. vagans C9-1), P. agglomerans E325, P. agglomerans P10c, Pseudomonas fluorescens A506, Gram-positive bacteria such as several Bacillus spp., among others Bacillus subtilis QST 713 (syn. B. amyloliquefaciens QST 713 or B. velezensis QST 713), B. subtilis BD170, and Lactobacillus plantarum, and fungi, such as Aureobasidium pullulans, and Metschnikowia pulcherrima, as well as bacteriophages, and non-virulent strains of E. amylovora which are continually found and characterized as possible control agents of fire blight (Johnson and Stockwell, 2000). Biological control agents can be as effective as antibiotics in the treatment of fire blight. The efficacy of several microbial products available on the market for control of fire blight was evaluated in the Eastern US. The treatments have been shown to have a moderate efficacy. The treatments with biological control agents reduced infection caused by E. amylovora from 9 to 36%, while values for the control with streptomycin ranged from 59 to 67% (Sundin et al, 2009).

The mechanism of action in various biopesticides against E. amylovora is the production of secondary metabolites with antimicrobial activity, such as pantocines or cyclolipopeptides, like in P. vagans C9-1, P. agglomerans E325, P. agglomerans P10c, B. subtilis QST 713 (syn. B. amyloliquefaciens QST 713 or B. velezensis QST 713). In some cases, these metabolites can be toxic (Ongena and Jacques, 2008). Another aspect of some biological control agents is their possible opportunistic pathogenicity of the species P. fluorescens and P. agglomerans which have been cited as causing clinical infections and sepsis which can be an obstacle to the authorization of strains of these species for use as a pesticide agent. In the EU, P. fluorescens A506, P. vagans C9-1, P. agglomerans E325 and P. agglomerans P10c have not been considered as QPS (“Qualified Presumption of Safety”), a term similar to the known GRAS (“Generally Recognized as Safe”), and therefore are not authorized for biological control of fire blight. Although biological products have advantages over chemical pesticides, environmental and host conditions may affect their biological activity, such that their effectiveness is generally variable and significantly lower than that of reference antibiotics (EP2998387A1). In the EU, biocontrol agents and corresponding products have been authorised according to Directive 128/2009 and Regulation 1107/2009, consisting of B. amyloliquefaciens QST 713 as contained in the commercial product Serenade Max, A. pullulans strains DSM14190 and DSM14191, and bacteriophages pEaH1 and pEaH2 (EU Pesticide database, online).

Serenade MAX (also termed Serenade or Serenade Max herein) is a biological control agent to aid in control and suppression of several plant diseases including fire blight. The active biological agent in Serenade products is Bacillus amyloliquefaciens strain QST 713. B. amyloliquefaciens QST 713 has the genetic capacity to produce the cyclic lipopeptides: iturin A, bacillomycin, fengycin and surfactin; the polyketides: macrolactin, bacillaene and difficidin; the iron siderophore bacillibactin; the antimicrobial dipeptide bacilysin, the antibiotics ericin A and ericin S, the volatile compound 2,3-butanediol; and the plant growth hormone indole-3-acetic acid. The secondary metabolites detected in Serenade products were iturin A, fengycin A, fengycin B, surfactin, bacillaene, difficidin, ericin A and ericin S (doi: 10.2903/j.efsa.2021.6381). It is known that iturins and surfactins are strong surfactants showing membrane damaging properties (lytic activity) in vitro and can therefore be regarded as secondary metabolites with antimicrobial activity. The view of European regulatory authorities is that based on the prescribed mode of application consumer exposure to toxic metabolites formed by B. amyloliquefaciens strain QST 713 cannot be excluded. Therefore, Serenade Max/B. amyloliquefaciens strain QST 713 was not proposed to be included in Annex IV of Regulation (EC) No 396/2005 which includes pesticide active substances that do not require a review of the existing maximum residue levels. In addition, high mortality was observed when Serenade was evaluated for toxicity, infectivity and pathogenicity in fish and aquatic invertebrates. Secondary metabolites were identified as one of the possible reasons for the observed mortality (doi: 10.2903/j.efsa.2021.6381). A recent study assessed potential harmful lethal and sublethal effects of the commercial plant protection product containing B. amyloliquefaciens QST 713 on honeybees. The study showed significantly lower expression of genes for Apidaecin, Defensin-1, and Hymenoptaecin in winter bees after 10 days of chronic exposure to B. amyloliquefaciens QST 713. Despite no direct lethal effect of the commercial plant protection product containing B. amyloliquefaciens QST 713 was observed in adult bees following chronic exposure, the decrease in some immunity parameters observed in tested winter bees may potentially impair bee colony health and survival (Sabo et al, 2020).

Given current obstacles and problems with existent biological control agents against fire blight, it would be advantageous to develop a new biocontrol agent for the prevention and/or treatment of fire blight which would be based on a microorganism that does not produce secondary metabolites with antibacterial/antifungal properties in its genome.

Paraburkholderia phytofirmans PsJN has been reported as a prominent and efficient plant growth-promoting endophyte (Ait Barka et al., 2000) and a promising biological control agent against plant pathogens (Miotto-Vilanova et al., 2016). P. phytofirmans strain PsJN is a naturally occurring Gram-negative rod-shaped, nonsporulating, and motile bacterium. It has been formerly grouped in Burkholderia species, but recently reclassified as a member of the genus Paraburkholderia which has been mostly reported to be associated with plants and to have biocontrol and bioremediation properties (Sawana et al., 2014; Eberl and Vandamme, 2016). P. phytofirmans PsJN was first isolated from surface-sterilized onion roots infected with the mycorrhizal fungus Glomus vesiculiferum (Frommel et al., 1991a; Sessitsch et al., 2005). P. phytofirmans PsJN has a positive effect on plant productivity by employing several mechanisms. These mechanisms act either directly by providing adequate plant nutrition, and producing plant hormones, or indirectly by reducing susceptibility to diseases (Yang et al., 2009; Andreolli et al., 2016). The bacterium is also able to decrease the ethylene level in host plants through the production of the 1-aminocyclopropane-1-carboxylate (ACC) deaminase enzyme (Glick et al., 2007). Moreover, it confers plants resistance against a broad spectrum of phytopathogens by the induction of plant-mediated resistance response in above-ground parts of plants (Miotto-Vilanova et al., 2016). The strain has been also shown to induce tolerance of plants to various abiotic stresses including high temperature, cold, drought, and salinity. P. phytofirmans PsJN has been shown to reduce the severity of Pierce's disease in grapevine, caused by a pathogenic bacterium Xyllela fastidiosa (Baccari et al, 2019).

Accordingly, the technical problem underlying the present invention is the provision of means and methods to prevent and/or control plant pests, in particular, to prevent and/or control fire blight infections in plants.

This technical problem is solved by the embodiments and items as provided herein and as specifically provided in the appended claims.

Thus, in its broadest embodiment, the present invention relates to means and methods comprising Paraburkholderia bacteria for use in preventing and/or controlling pests on a plant and/or on a temporary part of a plant and/or being located inside of a plant.

In the context of this invention it is documented that Paraburkholderia bacteria, preferably Paraburkholderia bacteria that belong to the species Paraburkholderia phytofirmans, most preferably bacteria of the strain Paraburkholderia phytofirmans PsJN, have superior activity against certain pests compared to previously described bacteria. This is surprising since the bacteria of the present invention in contrast to previously described bacteria are incapable of producing secondary metabolites with antimicrobial activity which are normally required to prevent and/or control pests (exemplary bacteria that produce such secondary metabolites with antimicrobial activity are plant pathogens Burkholderia plantarii and Burkholderia glumae (formerly known as Pseudomonas glumae) which produce tropolone and toxoflavin respectively, or Bacillus amyloliquefaciens strain QST 713 as contained in Serenade Max, which also produces secondary metabolites as described above). In the context of the present invention and as illustrated in the appended examples, the inventors have therefore surprisingly found that Paraburkholderia bacteria, in particular the strain P. phytofirmans PsJN (accessible in DSMZ Deutsche Sammlung von Mikroorganismen und Zellkulturen, accession number DSM 17436) showed superior activity against the plant pest fire blight (caused by Erwinia amylovora/E. amylovora) compared to Bacillus amyloliquefaciens strain QST 713 as is contained in Serenade Max which represents the current gold standard in the treatment of fire blight caused by E. amylovora. The appended examples show that experiments comprising P. phytofirmans PsJN in a tank mix with the adjuvant Break Thru S 301 (which is a surfactant, in particular a polyethersiloxane/polyether trisiloxane) at all three adjuvant doses applied (0.05%, 0.1% and 0.2% v/v) prevented and/or controlled E. amylovora on pear trees at least equally well compared to the commercially available and optimized biocontrol agent Serenade Max (FIGS. 1-3). According to the inventors' field trial results which are shown in Table 1 below as well as in the appended examples and figures, all P. phytofirmans PsJN treatments statistically outperformed the untreated control (“Untreated Check”) and were similar in efficacy to the reference (item) Serenade Max. The treatments comprising P. phytofirmans PsJN applied in a tank mix with Break Thru S 301 at 0.1% and 0.2% surprisingly even yielded numerically better protection (21.7% and 22.6% respectively) in terms of lower percentages of fruits damaged by E. amylovora, compared to Serenade Max (30.1%) (FIGS. 4-6).

TABLE 1
Efficacy of P. phytofirmans PsJN against Erwinia amylovora on flower clusters and fruit.

| Trt. | | | Appl. | | | 10 days post | | 18 days post | | 28 days post | | 40 days post | |
|---|---|---|---|---|---|---|---|---|---|---|---|---|---|
| No. | Treatment name | Rate | Code | 0 | | 1^{st} appl. | | 1^{st} appl. | | 1^{st} appl. | | 1^{st} appl. | |
| 1 | Untreated Check | | | 0 | a | 1.25 | a | 6.75 | a | 60.00 | a | 147.5 | b |
| | | | | | | (0.0%) | | (0.0%) | | (0.0%) | | | |
| 2 | P. phytofirmans | 0.833 | ABC | 0 | a | 0.00 | b | 3.50 | b | 33.50 | b | 182.5 | ab |
| | PsJN, Break Thru | 1/100 | | | | (100.0%) | | (48.1%) | | (44.2%) | | | |
| | S 301 0.05% | 1 | | | | | | | | | | | |
| 3 | P. phytofirmans | 0.833 | ABC | 0 | a | 0.00 | b | 3.25 | b | 39.75 | b | 235.0 | a |
| | PsJN, Break Thru | 1/100 | | | | (100.0%) | | (51.9%) | | (33.8%) | | | |
| | S 301 0.1% | 1 | | | | | | | | | | | |
| 4 | P. phytofirmans | 0.833 | ABC | 0 | a | 0.00 | b | 1.25 | c | 40.00 | b | 211.3 | a |
| | PsJN, Break Thru | 1/100 | | | | (100.0%) | | (81.5%) | | (33.3%) | | | |
| | S 301 0.2% | 1 | | | | | | | | | | | |
| 5 | Serenade Max | 4.0 | ABC | 0 | a | 0.00 | b | 1.25 | c | 40.50 | b | 206.3 | a |
| | | kg/ha | | | | (100.0%) | | (81.5%) | | (32.5%) | | | |

| | | | | | |
|---|---|---|---|---|---|
| LSD P = .05 | . | 0.345 | 1.089 | 13.237 | 43.90 |
| Standard Deviation | 0.0 | 0.224 | 0.707 | 8.592 | 28.49 |
| CV | 0.0 | 89.44 | 22.1 | 20.1 | 14.5 |
| Grand Mean | 0.0 | 0.250 | 3.200 | 42.750 | 196.50 |
| Levene's F\hat{ } | . | 0.45 | 1.804 | 1.282 | 2.278 |
| Levene's Prob(F) | . | 0.771 | 0.181 | 0.321 | 0.109 |
| Rank X2 | . | . | . | . | . |
| P(Rank X2) | . | . | . | . | . |
| Skewness\hat{ } | . | 1.8758* | 0.5437 | −0.3423 | −0.2908 |
| Kurtosis\hat{ } | . | 6.3489* | −0.4413 | 1.7574 | 0.8745 |
| Replicate F | 0.000 | 1.000 | 1.333 | 3.238 | 31.920 |
| Replicate Prob(F) | 1.0000 | 0.4262 | 0.3096 | 0.0605 | 0.0001 |
| Treatment F | 0.000 | 25.000 | 40.600 | 5.483 | 5.410 |
| Treatment Prob(F) | 1.0000 | 0.0001 | 0.0001 | 0.0095 | 0.0100 |

These results are unexpected and are rendered possible by the means and methods as disclosed herein. A person skilled in the art knows that Bacillus amyloliquefaciens strain QST 713 (formerly also known as Bacillus subtilis QST 713 or strain AQ 713 which is synonymous to QST 713) as contained in Serenade Max and P. phytofirmans PsJN represent two taxonomically different strains of bacteria each of which exhibits vastly different characteristics. The taxonomy of Bacillus amyloliquefaciens strain QST 713 is as follows: Bacteria (Domain)—Firmicutes (Phylum)—Bacilli (Class)—Bacillales (Order)—Bacillaceae (Family)—Bacillus (Genus)—Bacillus amyloliquefaciens (Species)—Bacillus amyloliquefaciens QST 713 (Strain). The taxonomy of Paraburkholderia phytofirmans PsJN (P. phytofirmans PsJN) is as follows: Bacteria (Domain)—Proteobacteria (Phylum)—Betaproteobacteria (Class)—Burkholderiales (Order)—Burkholderiaceae (Family)—Paraburkholderia (Genus)—Paraburkholderia phytofirmans (Species)—Paraburkholderia phytofirmans PsJN (Strain). Both bacterial strains taxonomically diverge early at the “Phylum” level, and it is surprising that both bacterial strains despite their taxonomical distance protect at least equally well against fire blight as caused by E. amylovora.

It is known that Bacillus amyloliquefaciens strain QST 713, as is contained in Serenade Max, as well as several other bacterial strains that have been described to be inhibitory towards E. amylovora, such as Bacillus spp., Streptomyces lydicus, Pseudomonas fluorescens, Pantoea spp, Paenibacillus brasilensis, Burkholderia plantarii and Burkholderia glumae (formerly known as Pseudomonas glumae) produce secondary metabolites with antimicrobial activity such as lipopeptides (see, e.g. U.S. Pat. Nos. 6,060,051; 6,103,228; 6,291,426; 6,417,163; and 6,638,910), which can have potentially adverse effects on consumers as well as on the environment (fish, aquatic invertebrates as described above). Accordingly, it is desired to employ in context of this invention a microorganism that does not produce secondary metabolites with antimicrobial activity. The invention also provides for the use of such microorganisms that fulfill this need. However, these secondary metabolites with antimicrobial activity are known to be (a) key element(s) of (the above) microbes' mode of action to prevent and/or control E. amylovora infection (as is the case for example for the two plant pathogens Burkholderia plantarii and Burkholderia glumae; see Mitchell, Acta Horticulturae 338, 219-22 (1993), Mitchell et al, Org. Biomol. Chem, 3, 3540-3543 (2005) and Mitchell et al, Phytochemistry, 69, 2704-2707 (2008) or Bacillus amyloliquefaciens strain QST 713, e.g. as contained in Serenade Max). Therefore, it is thought that E. amylovora infection can be particularly well prevented and/or controlled by microbial biocontrol agents which are able to produce such secondary metabolites with antimicrobial activity. P. phytofirmans PsJN does not produce such secondary metabolites with antimicrobial activity and therefore wass not thought in the prior art to appear to be particularly well suited for use in preventing and/or controlling E. amylovora on a plant and/or on a temporary part of a plant and/or being located inside of a plant.

The person skilled in the art furthermore knows that Paraburkholderia spp. (in particular Paraburkholderia phytofirmans PsJN) are a distinct species within the genus Burkholderia sensu lato (Bach et al., Genomics, 114, 398-408 (2022)). The genus Burkholderia sensu lato is divided into Burkholderia sensu stricto (s.s.) and six other genera named Paraburkholderia, Caballeronia, Robbsia, Mycetohabitans, Trinickia, and Pararobbsia. The genera Paraburkholderia, Caballeronia, and Trinickia contain plant symbionts. On the other hand, Robbsia are phytopathogens, Pararobbsia are environmental species, Mycetohabitans accommodates fungal endosymbionts, and Burkholderia s.s. are plant pathogens, including Burkholderia gladioli, Burkholderia glumae (formerly Pseudomonas glumae), and Burkholderia plantarii, and the opportunistic human and other animal pathogenic species, such as Burkholderia mallei and Burkholderia pseudomallei. Furthermore, Burkholderia glumae (formerly: Pseudomonas glumae) is known to secrete toxoflavin, which is known to induce bacterial wilt in many field crops (Jeong et al, Plant Disease, 87, 890-895 (2003)).

The mode of action of Bacillus amyloliquefaciens strain QST 713 as is contained in Serenade Max (as well as the mode of action of all other microbial biocontrol agents which are able to produce secondary metabolites with antimicrobial activity) is based on said secondary metabolites with antimicrobial activity which are known to act on the cell membrane of microbes (see, e.g., Fungicide Resistance Action Committee, FRAC-Code F6). The mode of action of Bacillus amyloliquefaciens strain QST 713 as is contained in Serenade Max is therefore a contact-based mode of action requiring the secondary metabolite(s) and the pest, in particular E. amylovora, to get in contact with one another. On the contrary, P. phytofirmans PsJN as employed herein is an endophytic bacterium which is incapable of producing secondary metabolites with antimicrobial activity which exerts its positive effects mainly from within a plant.

Bacillus amyloliquefaciens strain QST 713 as contained in Serenade Max is recommended to be applied to the whole aerial surface of a plant preferably comprising the leaves and the trunk of a plant as early as possible, preferably before the onset of a pathogenic infection. Such application thus ideally completely coats/covers the plant with Bacillus amyloliquefaciens strain QST 713 producing secondary metabolites with antimicrobial activity which creates a zone of inhibition where the bacteria have been applied and thus prevents attachment and penetration of the pests (in) to the plant in said zone of inhibition. Bacillus amyloliquefaciens strain QST 713 as is contained in Serenade Max therefore primarily achieves best results when applied preventatively and when complete coverage of the plant is achieved.

The inventors in the course of carrying out their experiments have surprisingly found that when P. phytofirmans PsJN in particular together with an adjuvant (i.e. a second agent, preferably a surfactant, more preferably the polyethersiloxane/polyether trisiloxane of the present invention) is specifically applied directly to flowers/flower clusters on plants of the Rosaceae family, particularly on pears early (BBCH 61) and during flowering period (BBCH 61-67), P. phytofirmans PsJN is able to effectively prevent and/or control fire blight caused by E. amylovora. The mode of application, in particular to the flowers during said flowering period, is speculated to have led to the even more surprising effect that P. phytofirmans PsJN does not only prevent and/or control E. amylovora infection on pear compared to an untreated control, but that the unoptimized compositions comprising the P. phytofirmans PsJN of the present invention can even achieve comparable results to Serenade Max in preventing and/or controlling pests on a plant, such as on a plant that belongs to the Rosaceae family of plants, such as in particular pear. This is unexpected since P. phytofirmans PsJN has never been used before to treat plants of the Rosaceae family of plants to prevent and/or control pests, such as in particular E. amylovora (which appears to be particularly susceptible to secondary metabolites with antimicrobial activity such as toxoflavin or tropolone (see e.g. Mitchell, Acta Horticulturae 338, 219-22 (1993), Mitchell et al, Org. Biomol. Chem, 3, 3540-3543 (2005) and Mitchell et al, Phytochemistry, 69, 2704-2707 (2008)), which P. phytofirmans PsJN does not produce) and since Serenade Max constitutes an optimized commercial product, which represents the current gold standard for treating fire blight caused by E. amylovora using biocontrol agents.

The inventors have surprisingly found that plants of the Rosaceae family, which constitutes a medium-sized family of flowering plants, represent a particularly preferred family of plants when P. phytofirmans PsJN is to be used as a biocontrol agent against E. amylovora to prevent and/or control fire blight. This is particularly the case in the context when a composition of said P. phytofirmans PsJN at a working concentration of 10⁸ CFU/ml (i.e. 10⁸ bacteria/ml) co-formulated with an adjuvant such as the preferred surfactant polyethersiloxane/polyether trisiloxane at a concentration of 0.1 or 0.2 volume percent in said composition is repeatedly applied to (contacted to) flowers of plants that belong to the Rosaceae family of plants. Applications may be carried out three times with two 10-day intervals during flowering period (BBCH 61-67) because during said period, in particular early in that period, plants that belong to the Rosaceae family of plants, in particular pear, display potentially very suitable entry sites, in particular flowers, for the endophyte P. phytofirmans PsJN into the plant.

While the specific mechanism is unknown, it is contemplated that the specifically co-formulated polyethersiloxane/polyether trisiloxane as is comprised in a particularly preferred composition of the present invention may be particularly effective when said composition is applied to (contacted to) flowers/petals of a plant that belongs to the Rosaceae family of plants such as pear with regard to the ability to retain P. phytofirmans PsJN on said plant and/or said flowers/petals of said plant. This is in particular shown in the appended examples which clearly indicate that there is a surfactant/adjuvant-dependent difference in the effectivity of P. phytofirmans PsJN as contained in the composition of the present invention to prevent and/or control fire blight infection in a plant of the Rosaceae family, in particular pear, when applied (contacted) to flowers three times during BBCH 61-67 at a concentration of 10⁸ bacteria/ml (i.e., at rate of 5×10¹³ bacteria per hectare). This is in particular the case when the surfactant is a polyethersiloxane/polyether trisiloxane at a concentration of 0.1-0.2 volume percent in the preferred composition comprising the P. phytofirmans PsJN of the present invention at a concentration of 10⁸ bacteria/ml.

Within its meaning as a biocontrol agent (i.e., to control and/or prevent pests on a plant and/or on a temporary part of a plant and/or being located inside of a plant) the effect of P. phytofirmans (PsJN) on pests has been assessed before in the context of grapevine, tomato, olive and Arabidopsis thaliana, which all do not belong to the Rosaceae family of plants. Thus, it is entirely unclear how the results obtained in these experiments translate to the treatment of pests on other plants such as plants of the Rosaceae family of plants.

Effects of bacteria of the genus (Para) Burkholderia were further assessed in context of switchgrass, maize, potato, ryegrass, quinoa, rapeseed (Brassica napus), wheat, hybrid poplar, rice, white clover, bamboo, barley, or sugarcane. The person skilled in the art knows that all of these plants do not belong to the Rosaceae family of plants. A person skilled in the art furthermore knows, that studies involving the assessment of the effect of Burkholderia, Paraburkholderia, Paraburkholderia phytofirmans and/or Paraburkholderia phytofirmans PsJN on plants, which are not infected with, to be infected with or at the risk of becoming infected with pests (i.e., plants that are at no point in time during assessment in contact with said pests) do not allow for any conclusions to be made in the search for a solution to the technical problem pertaining to the present invention, which relates to plants that are infected with, to be infected with or at the risk of becoming infected with pests. Said “risk of becoming infected with pests” is in a non-limiting manner inter alia defined by a homogeneous pest presence across a given field trial which may or may not be at a usual level of infestation for the given trial area for the given period of the year.

Where P. phytofirmans (PsJN) within its meaning as a biocontrol agent was employed to prevent and/or control pests, its impact on a diverse range of said pests was assessed. Pests that have been assessed in this context include multiple fungi such as Botrytis cinerea (B. cinereal), Verticillium dahliae (V. dahliae), Neofusicoccum parvum (N. parvum), Aspergillus niger (A. niger), Colletotrichum acutatum (C. acutatum), Rhizoctonia solani (R. solani) and Fusarium oxysporum (F. oxysporum) and bacteria such as Pseudomonas syringae (P. syringae), Xylella fastidiosa (X. fastidiosa), Ralstonia solanacearum (R. solanacearum), as well as Pythium aphanidermatum (P. aphanidermatum; oomycete), all of which may have different characteristics especially with regards to their pathogenic effect on plants as well as their susceptibility to P. phytofirmans (PsJN) treatment (i.e., the contacting of said plants with P. phytofirmans PsJN to prevent and/or control said pests on said plant and/or on a temporary part of said plant and/or being located inside of said plant) depending on their taxonomic/phylogenetic distance (to one another). It is shown herein that P. phytofirmans (PsJN) is not effective against a range of bacteria and fungi, in contrast to Bacillus amyloliquefaciens QST 713; see Example 16 and FIGS. 23 and 24.

The susceptibility of a given pest to P. phytofirmans (PsJN) treatment may also strongly depend on the route of P. phytofirmans (PsJN) application (via roots, stem puncturing, leaves or as is the case in the present invention via flowers) and how a given pest responds to the specific way of application. The person skilled in the art is aware that this applies to all pests and, in particular, also to E. amylovora, the causative agent of fire blight, irrespective if said pests are specifically listed in this disclosure or not and that studies involving P. phytofirmans (PsJN) treatment of pests other than E. amylovora, which also do not apply P. phytofirmans (PsJN) directly to flowers, are not of any relevance to the present invention. Preferred herein is the application of the bacteria to be used herein to flowers. In other words, it is preferred herein that the bacteria to be used herein (or the compositions comprising the same as described herein) are to be applied during flowering time. For example, the application can be performed at the beginning of flowering (e.g. at BBCH 61), at full flowering (e.g. when 40-50% flowers on main raceme open, older petals are falling (e.g. at BBCH 64-65) and/or at decline of flowering (e.g. with majority of petals fallen (e.g. at BBCH 67)). It is envisaged and even preferred herein that applications can be sequential, i.e. the application can be performed at the beginning of flowering (e.g. at BBCH 61), followed by application at full flowering (e.g. when 40-50% flowers on main raceme open, older petals are falling (e.g. at BBCH 64-65), followed by application at decline of flowering (e.g. with majority of petals fallen (e.g. at BBCH 67)). Between these applications, there can be an appropriate time interval in between, e.g. a 8-day interval, 9-day interval, a 10-day interval, a 11-day interval or 12-day interval. A 10-day interval is preferred.

The person skilled in the art also knows the taxonomy of all pests, irrespective of their non-limiting disclosure within the context of the present invention. The taxonomy of selected non-limiting pests listed in the present disclosure is as follows. The taxonomy of E. amylovora is as follows: Bacteria (Domain)—Proteobacteria (Phylum)—Gammaproteobacteria (Class)—Enterobacteriales (Order)—Erwiniaceae (Family)—Erwinia (Genus)—Erwinia amylovora (Species). The taxonomy of Botrytis cinerea (B. cinerea) is as follows: Eukarya (Domain)—Fungi (Kingdom)—Ascomycota (Phylum)—Pezizomycotina (Subdivision)—Leotiomycetes (Class)—Helotiales (Order)—Sclerotiniaceae (Family)—Botrytis (Genus)—Botrytis cinerea (Species). The taxonomy of Verticillium dahliae (V. dahliae) is as follows: Eukarya (Domain)—Fungi (Kingdom)—Ascomycota (Phylum)—Pezizomycotina (Subdivision)—Sordariomycetes (Class)—Hypocreomycetidae (Subclass)—Glomerellales (Order)—Plectosphaerellaceae (Family)—Verticillium (Genus)—Verticillium dahliae (Species). The taxonomy of Neofusicoccum parvum (N. parvum) is: Eukarya (Domain)—Fungi (Kingdom)—Ascomycota (Division)—Pezizomycotina (Subdivision)—Dothideomycetes (Class)—Botryosphaeriales (Order)—Botryosphaeriaceae (Family)—Neofusicoccum (Genus)—Neofusicoccum parvum (Species). The taxonomy of Aspergillus niger (A. niger) is: Fungi (Kingdom)—Ascomycota (Division)—Pezizomycotina (Subdivision)—Eurotiomycetes (Class)—Eurotiales (Order)—Aspergillaceae (Family)—Aspergillus (Genus)—Aspergillus niger. The taxonomy of Colletotrichum acutatum is: Fungi (Kingdom)—Ascomycota (Division)—Pezizomycotina (Subdivision)—Sordariomycetes (Class)—Glomerellales (Order)—Glomerellaceae (Family)—Colletotrichum (Genus)—Colletotrichum acutatum (Species). The taxonomy of Rhizoctonia solani is: Fungi (Kingdom)—Basidiomycota (Division)—Agaricomycotina (Subdivision)—Agaricomycetes (Class)—Cantharellales (Order)—Ceratobasidiaceae (Family)—Rhizoctonia (Genus)—Rhizoctonia solani (Species). The taxonomy of Fusarium oxysporum is: Fungi (Kingdom)—Ascomycota (Division)—Pezizomycotina (Subdivision)—Sordariomycetes (Class) Hypocreales (Order)—Nectriaceae (Family)—Fusarium (Genus)—Fusarium oxysporum (Species). The taxonomy of Pseudomonas syringae is: Bacteria (Domain)—Proteobacteria (Phylum)—Gammaproteobacteria (Class)—Pseudomonadales (Order)—Pseudomonadaceae (Family)—Pseudomonas (Genus)—Pseudomonas syringae (Species). The taxonomy of Xylella fastidiosa is: Bacteria (Domain)—Proteobacteria (Phylum)—Gammaproteobacteria (Class)—Xanthomonadales (Order)—Xanthomonadaceae (Family)—Xylella (Genus)—Xylella fastidiosa (Species). The taxonomy of Ralstonia solanacearum is: Bacteria (Domain)—Proteobacteria (Phylum)—Betaproteobacteria (Class)—Burkholderiales (Order)—Burkholderiaceae (Family)—Ralstonia (Genus)—Ralstonia solanacearum (Species). The taxonomy of Pythium aphanidermatum is: SAR (Clade)—Stramenopiles—Oomycota (Phylum) Pythiales (Order)—Pythiaceae (Family)—Pythium (Genus)—Pythium aphanidermatum (Species).

A person skilled in the art is aware that in particular the “Gammaproteobacteria” Class of bacteria contain about 250 genera, which makes it the most genera-rich taxon of prokaryotes. Said Class is composed of all Gram-negative microbes and it is the most phylogenetically and physiologically diverse Class of Proteobacteria. Said Class harbors microbes of different shapes (rods, curved rods, cocci, spirilla and filaments) and it includes free living bacteria, biofilm formers, commensals, as well as symbionts whose metabolism can vary significantly between each other (aerobic, anaerobic, chemolithoautotrophics, chemoorganotrophics, photoauthotrophs, heterotrophs). Members of the “Gammaproteobacteria” Class of bacteria such as inter alia the pests Erwinia amylovora, Pseudomonas syringae and Xylella fastidiosa for example, are therefore considered to be significantly different, which in particular relates to the mechanism of their pathogenic effect(s) on plants, in particular plants that belong to the Rosaceae family of plants, as well as in particular their susceptibility to P. phytofirmans (PsJN) treatment to prevent and/or control them/said pests on a plant and/or on a temporary part of a plant and/or being located inside of a plant. This is especially the case, because a person skilled in the art knows that Enterobacteriales as represented by E. amylovora, as well as Pseudomonadales as represented by P. syringae, as well as Xanthomonadales as represented by X. fastidiosa within the Class of “Gammaproteobacteria” are taxonomically/phylogenetically far apart from each other.

Because of this heterogeneity of pests that have been assessed in the context of P. phytofirmans PsJN treatment, it was not obvious that P. phytofirmans PsJN could be successfully used to prevent and/or control E. amylovora on a plant and/or on a temporary part of a plant and/or being located inside of a plant (in particular in the context of a plant that belongs to the Rosaceae family of plants). To name one specific example, P. phytofirmans PsJN treatment inter alia failed in the context of olive trees that were infected with X. fastidiosa.

Within its meaning as a biocontrol agent (i.e., to control and/or prevent pests on a plant and/or on a temporary part of a plant and/or being located inside of a plant), P. phytofirmans (PsJN) has been applied in vitro, via roots, nodal explants, leaves or via (trunk/stem) puncture. The present inventors have found that in particular the application to flowers in combination with a polyethersiloxane/polyether trisiloxane at a concentration of 0.1-0.2 volume percent yielded surprisingly effective biocontrol results in the context of Rosaceae plants being infected with or being at the risk of becoming infected with pests such as E. amylovora.

Accordingly, in one embodiment the present invention relates to Paraburkholderia bacteria for use in preventing and/or controlling pests on a plant and/or on a temporary part of a plant and/or being located inside of a plant. In one embodiment, the invention relates to a method for preventing and/or controlling pests on a plant, and/or on a temporary part of a plant and/or being located inside of a plant comprising contacting Paraburkholderia bacteria to said plant and/or to said temporary part of said plant and/or a locus for plant growth. In another embodiment, the invention relates to a composition for preventing and/or controlling pests on a plant and/or on a temporary part of a plant and/or being located inside of a plant comprising Paraburkholderia bacteria. In one specific embodiment, said Paraburkholderia bacteria as comprised in the means and methods of the present invention may comprise bacteria of the species Paraburkholderia phytofirmans, preferably wherein said Paraburkholderia phytofirmans comprises bacteria of the strain Paraburkholderia phytofirmans PsJN.

In accordance with this invention, the means and methods of the present invention may be employed to prevent and/or control pests, in particular, E. amylovora, on a plant and/or on a temporary part of a plant and/or being located inside of a plant, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN. Accordingly, the present invention relates to means and methods comprising Paraburkholderia bacteria for use in preventing and/or controlling the pest E. amylovora, which is known to cause the plant disease fire blight.

Thus, the present invention relates to bacteria of the genus Paraburkholderia. Bacteria in accordance with the means and methods of the present invention may in particular comprise bacteria of the species Paraburkholderia phytofirmans and may more preferably comprise bacteria of the strain Paraburkholderia phytofirmans PsJN (P. phytofirmans PsJN) as is also evident from the appended examples and figures. In a preferred embodiment of the present invention, mutant strains of P. phytofirmans PsJN may also be employed in the means and methods of the present invention to prevent and/or control pests, in particular E. amylovora. Said mutant strains may confer resistance to antibiotics, in particular to antibiotics such as streptomycin and/or oxytetracycline. Said mutants may differ from the wildtype P. phytofirmans PsJN strain in genomic DNA and/or RNA alterations which may be introduced by targeted or non-targeted DNA or RNA mutation(s). Said alterations may comprise substitution(s), deletion(s), insertion(s), transversion(s) and/or duplication(s) of (a) gene(s) in the bacterial DNA. In case of deletions, it may be desirable to induce (a) frameshift mutation(s) in the coding region of said gene(s), which lead(s) to (a) premature stop codon(s) and thus either to (a) defective or (an) absent gene product(s) (protein(s)). In general, such alterations may also comprise the introduction of (a) specific mutation(s) into said gene(s) that only change(s) the function or expression levels of the resulting gene product (protein). (A) Gene(s) to be altered as described in the above may preferably comprise rpsL encoding the 30S ribosomal protein S12. In some instances, it may be preferred to introduce/add an orthogonal gene into the bacterial DNA of a bacterial strain, i.e. a gene that is not naturally comprised in the DNA of said strain. A person skilled in the art is aware of means and methods to perform the above alterations and how to determine if the resulting bacterial strain is suitable to carry out the means and methods of the present invention. Non-limiting examples of performing said alterations include gene-editing via targeted nucleases such as CRISPR/Cas9, TALEN and zinc finger nucleases, the introduction of plasmids or random mutagenesis of the parent strain using chemical or physical mutagens and subsequent selection of progeny possessing the desired phenotype.

The term “control” as used herein, is with reference to an established pest infection and may be with regard to an infected plant and/or a defined area comprising said infected plant and means killing, reducing in numbers, and/or reducing growth, feeding or normal physiological development.

The term “prevent” as used herein, has the same meaning as “control” and is to be understood with reference to either (i) the spread of established pest infections from an infected plant to (an) uninfected plant(s) the latter of which may thus be considered at risk of becoming infected with pests or (ii) the new establishment of a pest infection. Both, (i) and (ii) may again be with reference to a defined area comprising the plants to which the Paraburkholderia bacteria of the present invention are to be applied (contacted) to. In the appended examples said “risk of becoming infected with pests” is in a non-limiting manner for example indicated by the homogeneous pest presence across the trial which may be at the usual infestation level for the trial area for the period of the year in which the trial was conducted. Both terms (i.e., “control” and “prevent”) are to be understood as a direct consequence of the effect of the application/contacting of the Paraburkholderia bacteria of the present invention to plants.

In accordance with the means and methods of the present invention, preventing and/or controlling pests may increase undamaged fruit productivity of a plant that was or is to be contacted with Paraburkholderia or a composition comprising said Paraburkholderia bacteria. The term “undamaged fruit productivity” is to be understood in comparison to a plant that was not contacted with Paraburkholderia bacteria or with a composition comprising said Paraburkholderia bacteria. A plant that was not contacted with Paraburkholderia bacteria or with a composition comprising said Paraburkholderia bacteria may thus constitute an untreated check or a reference. For example, in the appended examples and figures it is surprisingly shown that compositions comprising the Paraburkholderia of the present invention together with varying concentrations of the surfactant/adjuvant Break Thru S 301 (“Break Thru”; 0.05%, 0.1% and 0.2% v/v) when applied/contacted to pears reproducibly yielded lower numbers of infected fruits and lower percentages of infected fruits compared to an untreated check (reference) that was only contacted with water. In this context, fruits may be determined to be infected inter alia visually in BBCH stages 71-73, i.e., by observing that a given fruit is shriveled, dried out, black, brown, displays droplets of bacterial ooze, and/or displays spots of different color compared to the rest of the fruiting body.

Pest(s) within the means and methods of the present invention relates to microbes which may have harmful effects on plants. Pests which may have harmful effects on plants may comprise bacteria, fungi, oomycetes, and viruses, but bacteria, in particular E. amylovora, are preferred. Pests in general and E. amylovora in particular may induce the non-limiting harmful effects like inter alia water soaking of the floral receptacle, ovary, and peduncles of blossoms, falling of petals, shriveled tissues which turn black, droplets of bacterial ooze, a “Shepherd's Crook” upon wilting of the tip of the shoot(s), blackening along the mid-vein of shoot leaves, dead shoot leaves, cracks in the bark, sunken surface, black wood, black fruit, and shriveled fruit.

In accordance with the means and methods of this invention, a “plant” is a plant that belongs to the Rosaceae family of plants. The Rosaceae family of plants constitutes a medium-sized family of flowering plants and comprises pome fruits such as apple, pears, peaches, quinces, apricots, plums, cherries, raspberries, loquats, strawberries, rose hips, hawthorns, almonds and roses. Within the means and methods of this invention pears, peaches and apples are however particularly preferred among said plants of the Rosaceae family of plants. Thus in one specific embodiment, the present invention relates to Paraburkholderia bacteria for use in preventing and/or controlling E. amylovora on a plant and/or on a temporary part of a plant and/or being located inside of a plant, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; and wherein said plant belongs to the Rosaceae family of plants, preferably wherein said plant (that belongs to the Rosaceae family of plants) comprises pears, peaches and apples. In another context, the present invention also relates to a method for preventing and/or controlling E. amylovora on a plant, and/or on a temporary part of a plant and/or being located inside of a plant comprising applying Paraburkholderia bacteria to said plant and/or to said temporary part of said plant and/or a locus for plant growth, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; and wherein said plant belongs to the Rosaceae family of plants, preferably wherein said plant (that belongs to the Rosaceae family of plants) comprises pears, peaches and apples. In yet another context, the present invention relates to a composition for preventing and/or controlling E. amylovora on a plant and/or on a temporary part of a plant and/or being contained inside of a plant comprising Paraburkholderia bacteria, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; and wherein said plant belongs to the Rosaceae family of plants, preferably wherein said plant (that belongs to the Rosaceae family of plants) comprises pears, peaches and apples. It is to be understood, that where a specific reference is made to a fruit of a plant that belongs to the Rosaceae family of plants, the reference is also applied to the plant that belongs to the Rosaceae family itself. For example, where reference is made to an “apple” or “apples”, this also applies to an “apple tree” or “apple trees”.

As used herein and in accordance with the means and methods of the present invention, “a temporary part” of a plant may be flowers, leaves and fruits, preferably flowers or flowers and leaves in particular flowers or flowers and leaves of a plant that belongs to the Rosaceae family of plants. Flowers in the context of this invention may comprise peduncle, receptacle, sepal, petal, stamen, anther, pistil, stigma and/or ovary. In another embodiment, said temporary part of a plant may also comprise seeds.

In accordance with this invention, a locus for plant growth may be soil.

The terms “on a plant”, “on a temporary part of a plant” and “being located inside of a plant” as used herein are to be understood with respect to the specific spatial occurrence of a pest in context of said plant. “On a plant” describes the occurrence of pests on the surface of all parts of a plant that do not belong to the temporary part of a plant. Non-limiting examples of such parts may inter alia comprise the roots, the root hairs, the stem, the shoot, and the trunk. “On a temporary part of a plant” describes the occurrence of pests on or within said temporary part of a plant, in particular on or within flowers, leaves, and fruits, but seeds may also be included in certain aspects of the invention. “Being located inside of a plant” describes the occurrence of pests inside of all parts of a plant that do not belong to the temporary part of a plant. Non-limiting examples of said all parts of a plant may inter alia include plant tissues like the meristematic tissue, the permanent tissue, the simple permanent tissue, the parenchyma, the collenchyma, the sclerenchyma, the epidermis, the complex permanent tissue, the xylem, the phloem and/or the interstitial space.

In accordance with the means and methods of the present invention, Paraburkholderia bacteria or the composition(s) comprising said Paraburkholderia bacteria may be contacted (applied) to a plant and/or a temporary part of a plant and/or a locus for plant growth, preferably wherein said Paraburkholderia bacteria or said composition comprising said Paraburkholderia bacteria is contacted (applied) to the flowers of said plant. In a preferred embodiment, said Paraburkholderia bacteria or said composition comprising said Paraburkholderia bacteria is contacted (applied) to the flowers of said plant early during flowering (BBCH 61). The term “a temporary part of a plant” may also include multiple different temporary parts of a plant. Accordingly, the Paraburkholderia bacteria or the composition(s) comprising said Paraburkholderia bacteria may be applied (contacted) to aerial parts of a plant like flowers, leaves, seeds, and fruits, but flowers and leaves are preferred, wherein flowers, in particular during early flowering period (BBCH 61), are particularly preferred in the context of this invention. Accordingly, the means and methods of the present invention relate to Paraburkholderia bacteria or (a) composition(s) comprising said Paraburkholderia bacteria that are applied (contacted) to a plant and/or a temporary part of said plant, preferably wherein said Paraburkholderia bacteria or said composition comprising said Paraburkholderia bacteria is applied (contacted) to the flowers of said plant, more preferably wherein said Paraburkholderia bacteria or said composition comprising said Paraburkholderia bacteria is applied (contacted) to the flowers of said plant during early flowering period (BBCH 61).

In one specific embodiment, the present invention relates to Paraburkholderia bacteria or (a) composition(s) comprising Paraburkholderia bacteria for use in preventing and/or controlling pests, in particular, E. amylovora, on a plant and/or on a temporary part of a plant and/or being located inside of a plant, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; wherein said plant belongs to the Rosaceae family of plants, preferably wherein said plant (that belongs to the Rosaceae family of plants) comprises pears, peaches and apples; and preferably wherein said Paraburkholderia bacteria or said composition comprising said Paraburkholderia bacteria are applied (contacted) to the flowers of said plant, in particular during early flowering period (BBCH 61).

In this context, the present invention also specifically relates to a method for preventing and/or controlling pests, in particular, E. amylovora, on a plant, and/or on a temporary part of a plant and/or being located inside of a plant comprising applying (contacting) Paraburkholderia bacteria to said plant and/or to said temporary part of said plant and/or a locus for plant growth, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; wherein said plant belongs to the Rosaceae family of plants, preferably wherein said plant (that belongs to the Rosaceae family of plants) comprises pears, peaches and apples; and preferably wherein said Paraburkholderia bacteria or said composition comprising said Paraburkholderia bacteria are applied (contacted) to the flowers of said plant, in particular during early flowering period (BBCH 61).

In agreement with the means and methods of the present invention, applying (contacting) of Paraburkholderia bacteria or compositions comprising said Paraburkholderia bacteria to a plant and/or a temporary part of said plant and/or a locus for plant growth may be performed at least once, at least twice, at least three times, at least four times or at least five times. Said applying/contacting may be performed before, during and/or after flowering (phenological development stage), but during flowering may be preferred. When the compositions of the present invention are applied/contacted multiple times, applying/contacting may take place at intervals of from about 1 to about 28 days, of from about 1 to about 21 days, of from about 1 to about 14 days, of from about 1 to about 10 days, and/or of from about 1 to about 7 days, e,g at intervals of 1, 2, 3, 4, 5, 6, 7, 8, 9, 10, 11, 12, 13 or 14 days, in particular 7, 8, 9 or 10 days. A first interval may be the same as a second or further interval (e.g., 10 days followed by 10 days) or they may be different (e.g. 10 days followed by 7 days and the like). Therefore, as is also shown in the appended examples and figures, applying to/contacting flowers or flowers and leaves (three times with a 10-day interval)) during BBCH 61-67 may be preferred. A person skilled in the art knows, that the BBCH-scale is to be used to identify certain phenological development stages of plants, in particular the flowering stage of a plant.

In other embodiments, the Paraburkholderia-based compositions of the present invention are applied to plants and/or a temporary part of a plant and/or a locus for plant growth following the application of a fumigant. Fumigants can be applied by shank injection, generally a minimum of 8 inches (20 cm) below the soil surface. Liquid formulations of fumigants can also be applied through surface drip chemigation to move the fumigant to a depth of 8 inches (20 cm) or more below the soil surface. Treated soil beds are covered with a plastic tarp to retain the fumigant in the soil for several days. This is done before planting and allowed to air out prior to planting. The Paraburkholderia-based compositions described herein would be applied/contacted after such air-out period. In some instances, the fumigants are applied at a rate that is less than the rate recommended on the product label.

In accordance with the means and methods of the present invention, contacting (i.e., application) of the Paraburkholderia bacteria or of (a) composition(s) comprising said Paraburkholderia bacteria to a plant and/or to temporary parts of said plant and/or to a locus for plant growth, preferably to flowers or flowers and/or leaves may inter alia comprise spraying, watering and/or soaking the plant or temporary parts of said plant with a composition of the present invention comprising Paraburkholderia bacteria which is formulated as a bacterial suspension. Preferably, said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml as is also shown in the appended examples. In the context of this invention the term “bacteria/ml” may refer to a concentration ratio comprising both dead and live bacteria, but live bacteria may be preferred. In the context of live bacteria, the term “bacteria/ml” may also be expressed by the term “CFU/ml”. The person skilled in the art is aware of methods to determine live counts of bacteria and knows that CFU is equivalent to “colony forming units”.

In one embodiment and in accordance with the means and methods of the present invention Paraburkholderia bacteria are applied/contacted to a plant and/or a temporary part of a plant and/or a locus for plant growth at a rate of at least 10⁸, 10⁹, 10¹⁰, 10¹¹, 10¹², 10¹³, 10¹⁴, 10¹⁵ 10¹⁶, 10¹⁷ bacteria per hectare, preferably, wherein said Paraburkholderia bacteria are applied/contacted at a rate of about 5×10¹³ bacteria per hectare. In other words, in one preferred embodiment, Paraburkholderia bacteria may preferably be formulated in a composition of about 10⁸ bacteria/ml, wherein said composition is applied/contacted to a plant and/or a temporary part of a plant and/or a locus for plant growth, preferably to flowers or flowers and leaves of a plant, at a rate of about 5×10¹³ bacteria per hectare as is also indicated in the appended examples. It is evident for a person skilled in the art how the required rate is to be calculated per plant.

In accordance with the means and methods of the present invention a composition comprising Paraburkholderia bacteria further comprises at least a second agent and optionally secreted metabolites of said Paraburkholderia bacteria. In some embodiments, the composition is contacted/applied as a fermentation product that includes the Paraburkholderia bacteria, and, optionally, residual fermentation broth. In one embodiment, the fermentation product is composed substantially of Paraburkholderia cells.

The term “at least” in the context of said at least second agent as contained in a composition comprising Paraburkholderia bacteria of the present invention refers to one or more different entities of said at least second agent(s). Therefore, a multiplicity of said second agent(s) may be contained in said composition, but generally, 1, 2, 3, 4, 5, 6, 7, 8, 9, 10, 11, 12, 13, 14, or 15 of said entities may be contained.

The term “optionally secreted metabolites” as used within the means and methods of the present invention refers to organic compounds that may be secreted by the Paraburkholderia bacteria into the composition(s) of the present invention, like, i.e., into a seed/fermentation culture or a final tank mix that is to be applied/contacted to a plant. Secreted metabolites may comprise all organic chemical compounds that are specifically secreted by the Paraburkholderia bacteria but in certain aspects may also include metabolites that are released from bacterial cells as a consequence of cell death or cell disruption. Non-limiting examples may inter alia include alcohols, amino acids, nucleotides, antioxidants, organic acids, vitamins, and/or polyols. “Optionally secreted metabolites” are fundamentally different from “secondary metabolites with antimicrobial activity” as for example secreted by Bacillus subtilis QST 713 as is contained in Serenade Max since “optionally secreted metabolites” may not prevent and/or control pests on a plant and/or on a temporary part of a plant and/or being located inside of a plant.

The term “second agent” within the means and methods of the present invention and as used herein refers to any agent that can be formulated together with the Paraburkholderia bacteria of this invention and which may be commercially available. A second agent supports Paraburkholderia bacteria in their ability to prevent and/or control pests on a plant and/or on a temporary part of a plant and/or being located inside of a plant and/or is able to provide (an) additional beneficial effect(s) to a plant itself. Exemplary, non-limiting second agents may inter alia comprise inorganic, organic, ionic, non-ionic compounds, minerals, sugars, amino acids, auxins, growth stimulants, chemical and/or organic fertilizers, fungicides, miticides, nematicides, insecticides, defensive compounds against herbivores, and/or surfactants. A person skilled in the art knows which second agent is to be used to support Paraburkholderia bacteria and/or to provide (an) additional specific beneficial effect(s) to a plant.

When the composition is a dried product, second agents can comprise cryoprotectants, e.g. for freeze-drying. Exemplary such cryoprotectants are additives, such as saccharose, lactose, trehalose, skimmed milk or similar. The term “dried product” and “dried formulation” can be used interchangeably herein.

In a preferred embodiment said second agent is a surfactant. Surfactants in context of the means and method of this invention, comprise agents that may be co-formulated with Paraburkholderia bacteria in a composition comprising said Paraburkholderia bacteria to lower the surface tension of said composition so that the composition adheres better on the surface of a plant and/or a temporary part of a plant. The addition of such surfactants may have the effect of super-spreading which is to be understood as a process whereby the composition is distributed widely and rapidly, resulting in an evenly wetted plant target surface such as on a plant and/or on a temporary part of a plant. A person skilled in the art knows, that surfactants in this context may also be equivalently called “adjuvants”, such as the adjuvants Silwet L-77 (3-(8-methoxyoctoxy)propyl-methyl-bis(trimethylsilyloxy)silane), Polysorbate/Tween 80 (2-[2-[3,4-Bis(2-hydroxyethoxy)oxolan-2-yl]-2-(2-hydroxyethoxy) ethoxy]ethyl octadec-9-enoate)] or Break Thru S 301 (BREAK-THRU® S 301) as was employed in the composition(s) of the appended examples. The conventionally known Break Thru S 301 is a polyethersiloxane/polyether trisiloxane and is defined by the following chemical nomenclature: 3-[methyl-bis(trimethylsilyloxy)silyl]propan-1-ol; 2-methyloxirane; oxirane. As used herein, the terms “polyethersiloxane” and “polyether trisiloxane” can be used interchangeably. They can refer to the compound mentioned above with the chemical nomenclature: 3-[methyl-bis (trimethylsilyloxy)silyl]propan-1-ol; 2-methyloxirane; oxirane. Since the inventors could surprisingly achieve excellent effects within the means and methods of the present invention, in particular when compositions comprising Paraburkholderia bacteria together with said Break Thru S 301 were employed to specifically apply said compositions to flowers of plants that belong to the Rosaceae family of plants, in particular to pear, the use of polyethersiloxane/polyether trisiloxane(s) as a surfactant may be preferred, in particular 3-[methyl-bis(trimethylsilyloxy)silyl]propan-1-ol; 2-methyloxirane; oxirane may be a preferred surfactant in context of this invention.

Accordingly, in one specific embodiment, the present invention relates to Paraburkholderia bacteria for use in preventing and/or controlling pest, in particular, E. amylovora, on a plant and/or on a temporary part of a plant and/or being located inside of a plant, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; wherein said plant belongs to the Rosaceae family of plants, preferably wherein said plant (that belongs to the Rosaceae family of plants) comprises pears, peaches and apples; wherein said Paraburkholderia are preferably applied/contacted to the flowers of said plant; and wherein said Paraburkholderia bacteria are formulated in a composition comprising a surfactant, preferably a polyethersiloxane/polyether trisiloxane. In another specific embodiment, the present invention relates to a method for preventing and/or controlling pests, in particular, E. amylovora, on a plant, and/or on a temporary part of a plant and/or being located inside of a plant comprising applying/contacting Paraburkholderia bacteria to said plant and/or to said temporary part of said plant and/or a locus for plant growth, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; wherein said plant belongs to the Rosaceae family of plants, preferably wherein said plant (that belongs to the Rosaceae family of plants) comprises pears, peaches and apples; wherein said Paraburkholderia are preferably applied/contacted to the flowers of said plant; and wherein said Paraburkholderia bacteria are formulated in a composition comprising a surfactant, preferably a polyethersiloxane/polyether trisiloxane.

In yet further specific embodiment, the present invention relates to a composition for preventing and/or controlling pests, in particular, E. amylovora, on a plant and/or on a temporary part of a plant and/or being located inside of a plant comprising Paraburkholderia bacteria, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN; wherein said plant belongs to the Rosaceae family of plants, preferably wherein said plant (that belongs to the Rosaceae family of plants) comprises pears, peaches and apples; wherein said Paraburkholderia bacteria are preferably applied/contacted to the flowers of said plant; and wherein said composition further comprises a surfactant, preferably a polyethersiloxane/polyether trisiloxane.

Preferably, the bacterial strain is Paraburkholderia phytofirmans PsJN (accessible in DSMZ-Deutsche Sammlung von Mikroorganismen und Zellkulturen, accession number DSM 17436). Preferably, the plant is pear (Pyrus communis). Preferably, the pest is Erwinia amylovora (which is a causative agent of fire blight). Preferably, the surfactant is polyethersiloxane/polyether trisiloxane.

In a particularly preferred embodiment, the bacterial strain is Paraburkholderia phytofirmans PsJN (accessible in DSMZ-Deutsche Sammlung von Mikroorganismen und Zellkulturen, accession number DSM 17436), the plant is pear (Pyrus communis), the pest is Erwinia amylovora (which is a causative agent of fire blight) and the surfactant is polyethersiloxane/polyether trisiloxane.

Whenever preventing and/or controlling pests is mentioned herein, it is also meant/included that the thereby caused plant disease is to be prevented and/or controlled. For example, if the pest is Erwinia amylovora it is also meant/included that the thereby caused of fire blight is to be prevented and/or controlled. In this sense terms like “preventing and/or controlling pests” can be interchangeably used with “preventing and/or controlling plant disease”. For example, terms like “preventing and/or controlling Erwinia amylovora” can be interchangeably used with “preventing and/or controlling fire blight”.

In another embodiment, the surfactant within the means and methods of the present invention, may constitute about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of a composition comprising Paraburkholderia bacteria, preferably wherein said surfactant constitutes about 0.1-0.2 volume percent of said composition comprising said Paraburkholderia bacteria. Particularly preferred is wherein said surfactant constitutes about 0.1 volume percent of said composition comprising said Paraburkholderia bacteria. This is also evident from the appended examples and figures. These indicate that there appears to be a surfactant-dependent difference in the effectivity of P. phytofirmans PsJN as is contained in the means and methods of the present invention to prevent and/or control E. amylovora infection of a plant of the Rosaceae family, in particular pear, when applied/contacted to flowers three times during BBCH 61-67 at a concentration of 10⁸ bacteria/ml (i.e., at a rate of 5×10¹³ bacteria per hectare). It is to be understood that the intended volume percentages of the surfactant may deviate from the exact volume percentages and that a deviation of up to +/−5% (volume percent) may occur. Accordingly, within the means and methods of the present invention, a composition comprising Paraburkholderia bacteria at a concentration of 10⁸ bacteria/ml and a surfactant, preferably a polyethersiloxane/polyether trisiloxane, preferably wherein the polyethersiloxane/polyether trisiloxane is at a concentration of about 0.1-0.2 volume percent of said composition.

The composition for use herein (particularly when to be applied to the plants) preferably is an aqueous composition comprising, consisting essentially of or consisting of the bacteria, water (e.g. tap water) and adjuvant/surfactant (e.g. Break Thru as described herein, preferably wherein the surfactant is polyethersiloxane/polyether trisiloxane as described herein).

The present invention also relates to a composition, wherein said composition is a formulated product, preferably wherein said formulated product is a ready-to-use product or a product that is to be diluted with a suitable diluent prior to use. The term “formulated product” as used herein relates to a product comprising the Paraburkholderia bacteria of the present invention that is to be used for agricultural applications. Agricultural applications may comprise inter alia, (commercial) crop and/or fruit production as well as ornamental plants. The term “ready-to-use product” relates to a product comprising a composition of the Paraburkholderia bacteria of the present invention together with all second agents being contained in a final composition to be applied/contacted to a plant and/or a temporary part of a plant and/or a locus for plant growth. It is to be understood that hence, said Paraburkholderia bacteria and said second agents being contained in said final composition to be applied/contacted to a plant and/or a temporary part of a plant and/or a locus for plant growth are adjusted to their final working concentrations, may be directly applied/contacted to a plant and/or a temporary part of a plant and/or a locus for plant growth without modifying said composition. In other words, no diluents, (further) second agents and the like are to be added to a “ready-to-use product” before applying/contacting said “ready-to-use product” to a plant and/or a temporary part of a plant and/or a locus for plant growth. Where a suitable diluent may be added prior to use, i.e. prior to applying/contacting a composition comprising the Paraburkholderia bacteria of the present invention to a plant and/or a temporary part of a plant and/or a locus for plant growth, the composition may be a concentrated stock composition or a dried powder which may or may not comprise all second agents and Paraburkholderia bacteria of the ready-to-use product, however at a specifically higher concentration. Thus, said stock composition or said dried powder may be diluted with a suitable diluent to adjust the final concentrations of said second agents and Paraburkholderia bacteria prior to use. Exemplary, non-limiting dilution steps may be, inter alia, 1-fold, 2-fold, 3-fold, 4-fold, 5-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 250-fold, 300-fold, 350-fold, 400-fold, 450-fold, 500-fold, 600-fold, 700-fold, 800-fold, 900-fold, 1000-fold, 1500-fold, 2000-fold, 3000-fold, 4000-fold, 5000-fold, 6000-fold, 7000-fold, 8000-fold, 9000-fold, 10000-fold. A person skilled in the art knows how respective dilution steps are to be understood and carried out. Suitable diluents in this context may be water, physiological solutions (like, e.g. a water-based 0.9% w/v sodium chloride solution), buffers (like, e.g. phosphate-buffered saline; PBS), and/or (diluted) growth medium (like, e.g. MM9 containing glucose, ammonium ions, sulfate ions, potassium, magnesium, iron, and calcium, as is also described in the appended examples).

In accordance with the above, the present invention also relates to uses of Paraburkholderia bacteria for the preparation of a composition, wherein said composition can be a dried powder or a spraying agent. Further non-limiting compositions may comprise emulsifiable concentrate(s), (concentrated) solutions, flowable(s), invert emulsion(s), dust, granule(s), pellets), wettable powder(s), water-dispersible granule(s) and dry flowable(s). In one specific embodiment, the present invention relates to uses of Paraburkholderia bacteria for the preparation of a composition, wherein said composition can be a dried powder or a spraying agent, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.

The invention also relates to a kit comprising Paraburkholderia bacteria or the composition(s) of the present invention, preferably together with (an) instruction(s) regarding the use of the kit and/or the methods of the present invention. The kit may include a composition comprising the Paraburkholderia bacteria of the present invention or mutants thereof. The kit of the present invention in particular is to be employed for (commercial) agricultural applications such as crop and/or fruit production.

In a particular embodiment of the present invention, the kits (to be prepared in context) of this invention or the methods and uses of the invention may further comprise or be provided with instructions. For example, said instructions may guide the skilled person (how) to employ the kit of the invention in the methods and uses provided herein and in accordance with the present invention. Particularly, said instructions may provide guidance to use or apply the herein provided methods and/or uses. These instructions may also be a product label.

The kit (to be prepared in context) of this invention may further comprise substances/chemicals and/or equipment suitable/required for carrying out the methods and uses of this invention. For example, such substances/chemicals, and/or equipment are devices, second agents, reagents, solvents, diluents, and/or buffers for storing and/or preparing a formulated product of the present invention.

Compositions of the present invention may in general be obtained by culturing Paraburkholderia bacteria according to methods well known in the art and as specified in the appended examples. Conventional large-scale microbial culture processes include submerged fermentation, solid-state fermentation, or liquid surface culture. Fermentation is configured to obtain high levels of viable bacteria as determined by “colony forming units” (CFU) of Paraburkholderia bacteria. The bacterial cells and (secreted) metabolites in culture media resulting from fermentation may be used directly or concentrated by conventional industrial methods, such as centrifugation, tangential flow filtration, depth filtration, and evaporation. Fermentation broth and broth concentrate are both referred to herein as “fermentation products”. Compositions of the present invention include fermentation products. In some embodiments, the concentrated fermentation broth is washed, for example, via a diafiltration process, to remove residual fermentation broth and metabolites.

The fermentation broth or broth concentrate may be dried with or without the addition of carriers to generate a dried powder as may be contained in a product that is to be diluted with a suitable diluent prior to use. Drying may be achieved by using conventional drying processes or methods such as spray drying, freeze-drying, tray drying, fluidized bed drying, drum drying, or evaporation. Dry microbial biomass production is often desired because it provides a stable and potent form of the microorganisms for use as a biological control agent. Dry microbial biomass suitable for use in the biocontrol/biopesticide industry can be obtained through various methods, including lyophilization, spray drying, and drum drying. Lyophilization, also known as freeze-drying, involves freezing the microbial culture and then subjecting it to a high vacuum to remove the frozen water. This method results in a dry powder that is highly stable and can be stored at room temperature. Spray drying involves atomizing the microbial culture into a fine mist and then passing it through a hot air stream to remove the water. This method also produces a dry powder that is stable. Drum drying involves spreading the microbial culture onto a drum that is heated to remove the water. Additives can be added to dry microbial biomass to enhance its stability, shelf-life, and effectiveness. Some examples of additives include sugars, amino acids, vitamins, and minerals. These can be added to the microbial culture before the drying process to provide a food source for the microorganisms and help them survive the drying process. Additionally, protectants such as trehalose or glycerol can also be added to the biomass to prevent damage to the microorganisms during storage or during application. Other types of additives, such as surfactants, can be added to the biomass to improve its dispersibility and increase its effectiveness as a biopesticide. Additionally, encapsulation of the microorganisms with materials such as chitosan or alginate can provide extra protection to the microorganisms and improve their shelf-life. The viability of the microorganisms after drying. The number of viable bacteria in the dried form is assessed by serial dilution and colony counting. The number of viable bacteria in dried formulation in powder may be in the range of 10⁸ to 10¹² CFU/g, preferably between 10⁹ CFU/g and 10¹¹ CFU/g dried product. More preferably, the dried product contains from about 1×10¹¹ CFU/g to 4×10¹¹ CFU/g. The dried product can also contain about 10¹⁰ CFU/g. A lower number of viable bacteria is however less preferred.

It is preferred herein that the composition to be applied herein has a number of viable bacteria between 10⁶ to 10⁹ CFU/ml, e.g., 10⁷ to 10⁹ CFU/ml, or 10⁸ to 10⁹ CFU/ml, e.g. about 10⁶, 10⁷, 10⁸, 10⁹ CFU/ml. Preferred herein are about 10⁸ CFU/ml.

When a composition (e.g., a suspension) for application to the plants is prepared using a dried product as described herein (e.g., a powder) it is intended that the prepared composition has a number of viable bacteria between 10⁶ to 10⁹ CFU/ml as explained above.

Generally, the Paraburkholderia bacteria are to be used herein in an effective amount in preventing and/or controlling pests as described herein.

“Effective amount” as used herein can refer to an amount where the Paraburkholderia bacteria are more effective in preventing and/or controlling pests compared to an appropriate (negative) control (e.g., untreated plants and/or plants treated with water or a composition comprising water and, optionally surfactant, wherein the water or composition does not comprise Paraburkholderia bacteria). An “effective amount” as used herein can also refer to an amount where the Paraburkholderia bacteria are as effective or more effective in preventing and/or controlling pests compared to a positive control, e.g., Serenade Max, Bayer, containing Bacillus amyloliquefaciens QST 713), or compared to a corresponding product/composition containing Bacillus amyloliquefaciens, specifically containing Bacillus amyloliquefaciens QST 713, or an antibiotic, such as streptomycin.

For example, it is reported in the prior art that the treatments with biological control agents reduced infection caused by E. amylovora from 9 to 36%, while values for the control with streptomycin ranged from 59 to 67% (Sundin et al, 2009). As shown in the appended examples, Paraburkholderia bacteria, and strain PsJN in particular, reduced pest infection (as assessed by the (average) number or percentage of infected/damaged flowers/flower clusters, shoot and/or fruit (e.g. per tree) between 50-80% compared to the untreated control, i.e. to a similar extent as streptomycin, while simultaneously avoiding the use of antibiotic agents (like streptomycin) and/or avoiding the use of microorganisms (such as Bacillus amyloliquefaciens) producing or capable of producing antibiotics. Thus, in one aspect, an “effective amount” of Paraburkholderia bacteria as used herein can refer to an amount where the Paraburkholderia bacteria reduce or are capable of reducing pest infection (e.g. (as assessed by the) (average) number or percentage of infected/damaged flowers/flower clusters, shoot(s) and/or fruit(s) (e.g. per tree) between 50-80% compared to an appropriate (negative) control, e.g. untreated plants and/or plants treated with water. In another aspect, an “effective amount” of Paraburkholderia bacteria as used herein can refer to an amount where the Paraburkholderia bacteria reduce or are capable of reducing pest infection (e.g. (as assessed by the) (average) number or percentage of infected/damaged flowers/flower clusters, shoot(s) and/or fruit(s) (e.g., per tree) to similar, same or increased extent as an antibiotic (e.g. streptomycin).

The effective amount of the Paraburkholderia bacteria to be used herein can, in view of the guidance provided herein, be readily determined by a skilled person in the art.

An “effective amount” of the Paraburkholderia bacteria to be used herein is, for example, if the composition to be applied herein has a number of viable bacteria between 10⁶ to 10⁹ CFU/ml, e.g. 10⁷ to 10⁹ CFU/ml, or 10⁸ to 10⁹ CFU/ml, e.g. about 10⁶·10⁷, 10⁸, 10⁹ CFU/ml. Preferred herein are about 10⁸ CFU/ml. When a composition (e.g., a suspension) for application to the plants is prepared using a dried product as described herein (e.g., a powder) it is intended that the prepared composition has a number of viable bacteria (an “effective amount”) between 10⁶ to 10⁹ CFU/ml as explained above.

An “effective amount” of the Paraburkholderia bacteria to be used herein is in the range of 10⁸ to 10¹² CFU/g, preferably between 10⁹ CFU/g and 10¹¹ CFU/g, more preferably, from about 1×10¹¹ CFU/g to 4×10¹¹ CFU/g, when the composition is a dried product, e.g. powder of the Paraburkholderia bacteria (i.e. when the product is Paraburkholderia bacteria powder as described herein). An “effective amount” of the Paraburkholderia bacteria to be used herein can also be about 10¹⁰ CFU/g, when the composition is a dried product.

A composition (e.g. suspension) of e.g. about 10⁶, 10⁷ or 10⁸ CFU/mL in water to be used/applied corresponds to 5×10¹¹, 5×10¹², or 5×10¹³ CFU/ha (5×10¹¹, 5×10¹² or 5×10¹³ bacteria per hectare) of P. phytofirmans PsJN to be applied.

The term “effective amount” of the Paraburkholderia bacteria or the like as used herein refers, in particular, to a level/concentration/amount of the bacteria sufficient to prevent and/or control pests as described herein. For example, such a level/concentration/amount is an amount where the Paraburkholderia bacteria are more effective in preventing and/or controlling pests compared to an appropriate control (e.g. untreated plants and/or plants treated with water or a composition comprising water and, optionally surfactant, wherein the water or composition does not comprise Paraburkholderia bacteria) or an amount where the Paraburkholderia bacteria are as effective or more effective in preventing and/or controlling pests compared to a positive control, e.g. Serenade Max, Bayer, containing Bacillus amyloliquefaciens QST 713), or compared to a corresponding product/composition containing Bacillus amyloliquefaciens, specifically containing Bacillus amyloliquefaciens QST 713. Exemplary “effective amounts” are described herein.

Paraburkholderia bacteria are, for example, effective in preventing and/or controlling pests, if the (average) number or percentage of infected plant parts (particularly flowers/flower clusters, shoot(s) and/or fruits) per plant (if the plant(s) is/are treated with Paraburkholderia bacteria plants and/or a composition comprising the same) is decreased compared to an appropriate control (e.g. (average) number or percentage of infected plant parts (particularly flowers/flower clusters, shoot(s) and/or fruits) per plant (if the plant(s) is/are untreated plants and/or plants treated with water or a composition comprising water and, optionally surfactant, wherein the water or composition does not comprise Paraburkholderia bacteria) or is the similar, the same or decreased compared to a positive control, e.g. if the plant(s) is/are treated with Serenade Max, Bayer, containing Bacillus amyloliquefaciens QST 713), or compared to a corresponding product/composition containing Bacillus amyloliquefaciens, specifically containing Bacillus amyloliquefaciens QST 713. The term “infected plant part” includes plant parts (particularly flowers/flower clusters, shoot(s) damaged by the infection, e.g. the infection might already have occurred and be no longer present, but the damage caused by the infection can still be assessed. Of course, plant parts damaged by the infection can also be assessed when the infection still is ongoing.

The resulting dry products may be further processed, such as by milling or granulation, to achieve a specific particle size or physical format. Carriers, such as liquid materials such as water, oil, and other organic or inorganic solvents and solid materials such as minerals, polymers, or polymer complexes derived biologically or by chemical synthesis may also be added post-drying.

Generally, the Paraburkholderia bacteria to be used in accordance with the present invention can be used in any form. For example, the Paraburkholderia bacteria (as well as a population thereof) might be packaged (for sale) as dry powder or in storage buffer. For example, the bacteria (e.g., bacteria powder) can also be encapsulated, for example the bacteria are prepared by spray-drying. The encapsulation might also be a formulation of the bacteria in a water-soluble shell or layer, e.g., which might dissolve when contacted with whether, for example, when the final composition for application to the plants is prepared when the dried product is dissolved in water/in an aqueous solution.

In a preferred aspect, Paraburkholderia bacteria (or population thereof) can be formulated/used (and hence packaged/sold) as dried powder. In other word, the Paraburkholderia bacteria are Paraburkholderia bacteria powder. It is understood that for preparing the composition to be applied to the plant, this powder is to be dissolved appropriately, preferably in water, prior to applying the Paraburkholderia bacteria (or the composition comprising the same) to the pests and/or plants. Thus, the Paraburkholderia bacteria (or the composition comprising the same) can be applied as a (liquid) spray (e.g. spray suspension). As described herein, the powder can be prepared, e.g., by (a method comprising) freeze-drying the bacteria. The composition to be applied can be a suspension, i.e., the bacteria are suspended appropriately e.g. in water, and optionally, a surfactant is added.

As described herein, the Paraburkholderia bacteria (as well as a population thereof), can be used as a pesticide, specifically a pesticide against Erwinia amylovora.

The pest stage at each application preferably is pre-infection.

The compositions can be applied with routine sprayers, e.g., with a back-mounted research lance sprayer at e.g. 8 bars and e.g. 600 litres of bacterial suspension per hectare e.g., in the first application, and 800 litres of bacterial suspension per hectare e.g. in the second and/or e.g. third application.

A composition (e.g., suspension) of e.g. about 10⁶, 10⁷ or 10⁸ CFU/mL in water to be used/applied corresponds to 5×10¹¹, 5×10¹², or 5×10¹³ CFU/ha (5×10¹¹, 5×10¹² or 5×10¹³ bacteria per hectare) of P. phytofirmans PsJN to be applied.

Generally, the Paraburkholderia bacteria are to be used herein in an effective amount in preventing and/or controlling pests as described herein. “Effective” as used herein can refer to an amount where the Paraburkholderia bacteria are more effective in preventing and/or controlling pests compared to an appropriate control (e.g., untreated plants and/or plants treated with water or a composition comprising water and, optionally surfactant, wherein the water or composition does not comprise Paraburkholderia bacteria). “Effective” as used herein can also refer to an amount where the Paraburkholderia bacteria are as effective or more effective in preventing and/or controlling pests compared to a positive control, e.g., Serenade Max, Bayer, containing Bacillus amyloliquefaciens QST 713), or compared to a corresponding product/composition containing Bacillus amyloliquefaciens, specifically containing Bacillus amyloliquefaciens QST 713.

The effective amount of the Paraburkholderia bacteria to be used herein can, in view of the guidance provided herein, be readily determined by a skilled person in the art.

Further embodiments are exemplified in the scientific part. The appended figures provide illustrations of the present invention. Whereas the experimental data in the examples and as illustrated in the appended figures are not considered to be limiting. The technical information comprised therein forms part of this invention. The invention thus also covers all further features shown in the figures individually, although they may not have been described in the previous or following description. Also, single alternatives of the embodiments described in the figures and the description and single alternatives of features thereof can be disclaimed from the subject matter of the other aspect of the invention.

### THE FIGURES SHOW

FIG. 1: Bar chart showing the incidence of Erwinia amylovora infection on flower clusters of pear trees (Pyrus communis) at 0, 10, 18, 28 DAF (days post first application) from left to right. Different letters (a, b, c) mean significant difference between treatment results.

FIG. 2: Incidence of Erwinia amylovora infection on flower clusters of pear trees. There is an overlap of data for the treatments P. phytofirmans PsJN with Break Thru 0.2%, P. phytofirmans PsJN with Break Thru 0.1%, and Serenade Max. FIG. 2 shows the same data as FIG. 1.

FIG. 3: Efficacy of P. phytofirmans PsJN against E. amylovora on flower clusters at 10, 18, 28 DAF (days post first application) from left to right.

FIG. 4: Incidence of Erwinia amylovora infection on fruit at 40 days after the first application. Total number of fruits per tree. Different letters (a, b, c) mean significant difference between treatment results.

FIG. 5: Incidence of Erwinia amylovora infection on fruit at 40 days after the first application. Number of diseased fruits per tree is shown.

FIG. 6: Incidence of Erwinia amylovora infection on fruit at 40 days after the first application. Percent of infected fruits per tree is shown.

FIG. 7: Infection with fire blight in flowers of untreated control pear tree.

FIG. 8: Infection with fire blight in fruits of untreated control pear tree.

FIG. 9: Healthy fruits in pear trees treated with P. phytofirmans PsJN with 0.05% v/v Break Thru S 301.

FIG. 10: Healthy fruits in pear trees treated with P. phytofirmans PsJN with 0.1% v/v Break Thru S 301.

FIG. 11: Healthy fruits in pear trees treated with P. phytofirmans PsJN with 0.2% v/v Break Thru S 301.

FIG. 12. Number of diseased flower clusters at 29 days after the first application (DAF).

FIG. 23 . . . . Efficacy of P. phytofirmans PsJN against Erwinia amylovora on flower clusters (%). Efficacy was calculated with the transformation Abbott.

FIG. 14. Number of diseased shoots at harvest at 125 days after the first application (DAF).

FIG. 15. Efficacy of P. phytofirmans PsJN against Erwinia amylovora on shoots at harvest at 125 days after the first application (DAF). Efficacy (%) was calculated with the transformation Abbott.

FIG. 16. Number of fruits per tree at harvest at 125 days after the first application (DAF).

FIG. 17. Number of damaged fruits per tree at 50 days after the first application (DAF).

FIG. 18. Percent of damaged fruits per tree at 50 days the first application (DAF).

FIG. 19 Efficacy on fruits (%) at 50 days after the first application (DAF). Efficacy was calculated with the transformation Abbott.

FIG. 20. Yield of fruits in kg per tree at harvest (125 days after the first application (DAF)).

FIG. 21. Yield of fruits in tonnes per hectare at harvest (125 days after the first application (DAF)).

FIG. 22. Total number of fruits per tree at harvest (125 days after the first application (DAF)).

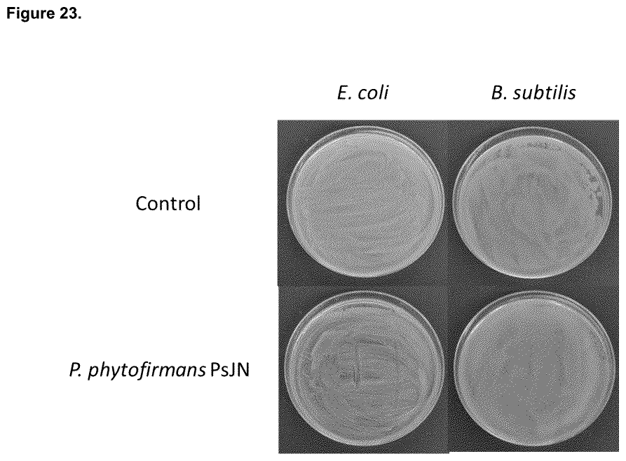

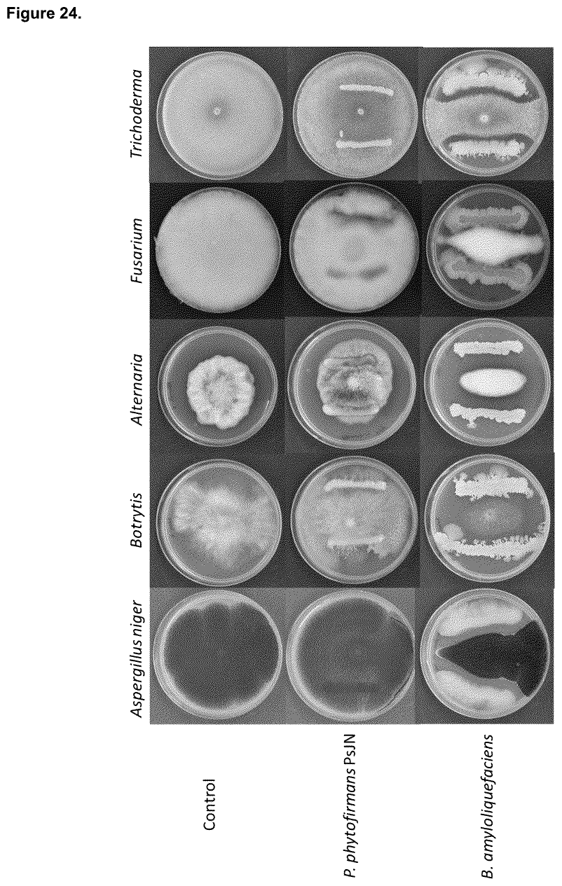

FIG. 23. Test of antimicrobial activity of P. phytofirmans PsJN. Top row: control plates; bottom row: No inhibition in P. phytofirmans swab tested against E. coli (left) and B. subtilis (right).

FIG. 24. Test of antimicrobial activity of P. phytofirmans PsJN and B. amyloliquefaciens QST 713 against fungi. First row: control. Middle row: P. phytofirmans PsJN-no inhibition. Bottom row: B. amyloliquefaciens-inhibition of fungi.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The methods and techniques of the present invention are generally performed according to conventional methods well known in the art and as described in various general and more specific references that are cited and discussed throughout the present specification unless otherwise indicated. See, e.g., Hankin and Peters, Snyder & Champness Molecular Genetics of Bacteria, 5th ed., Wiley (2020); Madigan et al., Brock Biology of Microorganisms, 15th ed., Pearson (2018); Helyer et al., Biological control in plant protection: a color handbook, 2nd ed., John Wiley & Sons (2010).

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the afore or following description. Also, single alternatives of the embodiments described in the figures and the description and single alternatives of features thereof can be disclaimed from the subject matter of the other aspect of the invention.

Furthermore, in the claims, the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

In this specification, it is to be understood that “means and methods” (of the present invention) specifically relates to (i) the Paraburkholderia bacteria as may be employed in the context of this invention, (ii) the methods and uses comprising Paraburkholderia bacteria as employed in the context of this invention and (iii) the compositions comprising Paraburkholderia bacteria as may be employed in the context of this invention. Thus, whenever reference is made to “means and methods” (of the present invention) this applies to all of the categories (i-iii) in the above.

In this specification, a number of documents including scientific publications, patent applications, and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

As used herein, the terms “comprising”, “including”, “having” or grammatical variants thereof are to be taken as specifying the stated features, integers, steps or components but do not preclude the addition of one or more additional features, integers, steps, components or groups thereof. The terms “comprising”/“including”/“having” encompass the terms “consisting of” and “consisting essentially of”. Thus, whenever the terms “comprising”/“including”/“having” are used herein, they can be replaced by “consisting essentially of” or, preferably, by “consisting of”.

The terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed product, composition, device or method and the like.

Thus, the term “consisting essentially of” means that specific further components (or likewise features, integers, steps and the like) can be present, namely those not materially affecting the essential characteristics of the product, composition, device or method. In other words, the term “consisting essentially of” (which can be interchangeably used herein with the term “comprising substantially”), allows the presence of other components in the product, composition, device or method in addition to the mandatory components (or likewise features, integers, steps and the like), provided that the essential characteristics of the product, composition, device or method are not materially affected by the presence of other components.

As used herein the term “about” refers to ±25%, preferably ±20%, ±15%, ±10%, more preferably ±5%.

As used herein, “a” or “an” may mean one or more.

In accordance with the above, the present invention relates to, inter alia, the following items:
1. Paraburkholderia bacteria for use in preventing and/or controlling pests on a plant and/or on a temporary part of a plant and/or being located inside of a plant.
2. Paraburkholderia bacteria for use according to item 1, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.
3. Paraburkholderia bacteria for use according to items 1 and 2, wherein said preventing and/or controlling of pests increases undamaged fruit productivity of said plant.
4. Paraburkholderia bacteria for use according to any one of items 1 to 3, wherein said pests comprise Erwinia amylovora.
5. Paraburkholderia bacteria for use according to any one of items 1 to 4, wherein said plant belongs to the Rosaceae family of plants.
6. Paraburkholderia bacteria for use according to item 5, wherein said Rosaceae family of plants comprises pears, peaches, apples, quinces, apricots, plums, cherries, raspberries, loquats, strawberries, rose hips, hawthorns, almonds, and roses, preferably wherein said Rosaceae family of plants comprises pears, peaches and apples.
7. Paraburkholderia bacteria for use according to any one of items 1 to 6, wherein said temporary part of said plant is selected from the group consisting of flowers, leaves, and fruits.
8. Paraburkholderia bacteria for use according to any one of items 1 to 7, wherein said Paraburkholderia bacteria are formulated in a composition, preferably wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.
9. Paraburkholderia bacteria for use according to item 8, wherein said composition further comprises at least a second agent and optionally secreted metabolites of said Paraburkholderia bacteria.
10. Paraburkholderia bacteria for use according to item 9, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores preferably wherein said at least second agent is a surfactant.
11. Paraburkholderia bacteria for use according to item 10, wherein said surfactant is a polyethersiloxane/polyether trisiloxane.
12. Paraburkholderia bacteria for use according to item 10 and 11, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.
13. Paraburkholderia bacteria for use according to any one of items 1 to 12, wherein said Paraburkholderia bacteria or said composition comprising said Paraburkholderia bacteria is contacted to said plant and/or said temporary part of said plant, preferably wherein said Paraburkholderia bacteria or said composition comprising said Paraburkholderia bacteria is contacted to the flowers of said plant.
14. Paraburkholderia bacteria for use according to item 13, wherein said contacting is performed at least once, at least twice, at least three times, at least four times or at least five times.
15. Paraburkholderia bacteria for use according to items 13 and 14, wherein said Paraburkholderia bacteria are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein said Paraburkholderia bacteria are contacted at a rate of about 5×10¹³ bacteria per hectare.
16. A method for preventing and/or controlling pests on a plant, and/or on a temporary part of a plant and/or being located inside of a plant comprising contacting Paraburkholderia bacteria to said plant and/or to said temporary part of said plant and/or a locus for plant growth.
17. The method of item 16, wherein said Paraburkholderia bacteria comprises bacteria of the species Paraburkholderia phytofirmans, preferably wherein said Paraburkholderia phytofirmans comprises bacteria of the strain Paraburkholderia phytofirmans PsJN.
18. The method of items 16 and 17, wherein said preventing and/or controlling of pests increases undamaged fruit productivity of said plant.
19. The method of any one of items 16 to 18, wherein said pests comprise Erwinia amylovora.
20. The method of any one of items 16 to 19, wherein said plant belongs to the Rosaceae family of plants.
21. The method of item 20, wherein said Rosaceae family of plants comprises pears, peaches, apples, quinces, apricots, plums, cherries, raspberries, loquats, strawberries, rose hips, hawthorns, almonds, and roses, preferably wherein said Rosaceae family of plants comprises pears, peaches and apples.
22. The method of any one of items 16 to 21, wherein said temporary part of said plant is selected from the group consisting of flowers, leaves and fruits.
23. The method of any one of items 16 to 22, wherein said Paraburkholderia bacteria are formulated in a composition, preferably wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.
24. The method of item 23, wherein said composition further comprises at least a second agent and optionally secreted metabolites of said Paraburkholderia bacteria.
25. The method of item 24, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores preferably wherein said at least second agent is a surfactant.
26. The method of item 25, wherein said surfactant is a polyethersiloxane/polyether trisiloxane.
27. The method of items 25 and 26, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.
28. The method of any one of items 16 to 27, wherein said Paraburkholderia bacteria or said composition comprising said Paraburkholderia bacteria is contacted to said plant and/or said temporary part of said plant and/or said locus for plant growth, preferably wherein said Paraburkholderia bacteria or said composition comprising said Paraburkholderia bacteria is contacted to the flowers of said plant.
29. The method of item 28, wherein said contacting is performed at least once, at least twice, at least three times, at least four times or at least five times.
30. The method of items 28 and 29, wherein Paraburkholderia bacteria are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein Paraburkholderia bacteria are contacted at a rate of about 5×10¹³ bacteria per hectare.
31. The method of any one of items 16 to 30, wherein said locus for plant growth is soil.
32. A composition for preventing and/or controlling pests on a plant and/or on a temporary part of a plant and/or being located inside of a plant comprising Paraburkholderia bacteria.
33. The composition of item 32, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.
34. The composition of items 32 and 33, wherein said preventing and/or controlling of pests increases undamaged fruit productivity of said plant.
35. The composition of any one of items 32 to 34, wherein said pests comprise Erwinia amylovora.
36. The composition of any one of items 32 to 35, wherein said plant belongs to the Rosaceae family of plants.
37. The composition of item 36, wherein said Rosaceae family of plants comprises pears, peaches, apples, quinces, apricots, plums, cherries, raspberries, loquats, strawberries, rose hips, hawthorns, almonds, and roses, preferably wherein said Rosaceae family of plants comprises pears, peaches and apples.
38. The composition of any one of items 32 to 37, wherein said temporary part of said plant is selected from the group consisting of flowers, leaves and fruits.
39. The composition of any one of items 32 to 38, wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.
40. The composition of any one of items 32 to 39, wherein said composition further comprises at least a second agent and optionally secreted metabolites of said Paraburkholderia bacteria.
41. The composition of item 40, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores preferably wherein said at least second agent is a surfactant.
42. The composition of item 41, wherein said surfactant is a polyethersiloxane/polyether trisiloxane.
43. The composition of items 41 and 42, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.
44. The composition of any one of items 32 to 43, wherein said composition is contacted to said plant and/or said temporary part of said plant, preferably wherein said composition is contacted to the flowers of said plant.
45. The composition of item 44, wherein said contacting is performed at least once, at least twice, at least three times, at least four times or at least five times.
46. The composition of items 44 and 45, wherein Paraburkholderia bacteria being contained in said composition are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein Paraburkholderia bacteria are contacted at a rate of about 5×10¹³ bacteria per hectare.
47. The composition of any one of items 32 to 46, wherein said composition is a formulated product, preferably wherein said formulated product is a ready-to-use product or a product that is to be diluted with a suitable diluent prior to use.
48. A kit comprising the composition of item 47, preferably together with instructions regarding the use of the method of any one of items 16 to 31.
49. Use of Paraburkholderia bacteria for the preparation of a composition, wherein said composition can be a dried powder or a spraying agent.
50. The use of Paraburkholderia bacteria according to item 49, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.

Furthermore, the present invention also relates to the following items:
1. Paraburkholderia bacteria for use in preventing and/or controlling Erwinia amylovora on a plant and/or on a temporary part of a plant and/or being located inside of a plant.
2. Paraburkholderia bacteria for use according to item 1, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.
3. Paraburkholderia bacteria for use according to item 1 and 2, wherein said preventing and/or controlling of pests increases undamaged fruit productivity of said plant.
4. Paraburkholderia bacteria for use according to any one of items 1 to 3, wherein said plant belongs to the Rosaceae family of plants, wherein said Rosaceae family of plants preferably comprises pears, peaches, apples, quinces, apricots, plums, cherries, raspberries, loquats, strawberries, rose hips, hawthorns, almonds, and roses, wherein said Rosaceae family of plants more preferably comprises pears, peaches and apples.
5. Paraburkholderia bacteria for use according to any one of items 1 to 4, wherein said temporary part of said plant is selected from the group consisting of flowers, leaves and fruits.
6. Paraburkholderia bacteria for use according to any one of items 1 to 5, wherein said Paraburkholderia bacteria are formulated in a composition, preferably wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.
7. Paraburkholderia bacteria for use according to item 6, wherein said composition further comprises at least a second agent and optionally secreted metabolites of said Paraburkholderia bacteria.
8. Paraburkholderia bacteria for use according to item 7, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores, preferably wherein said at least second agent is a surfactant, more preferably wherein said surfactant is a polyethersiloxane/polyether trisiloxane.
9. Paraburkholderia bacteria for use according to item 8, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.
10. Paraburkholderia bacteria for use according to any one of items 1 to 9, wherein said Paraburkholderia bacteria or said composition comprising said Paraburkholderia bacteria is contacted to said plant and/or said temporary part of said plant, preferably wherein said Paraburkholderia bacteria or said composition comprising said Paraburkholderia bacteria is contacted to the flowers of said plant.
11. Paraburkholderia bacteria for use according to item 10, wherein said contacting is performed at least once, at least twice, at least three times, at least four times or at least five times.
12. Paraburkholderia bacteria for use according to items 10 and 11, wherein said Paraburkholderia bacteria are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein said Paraburkholderia bacteria are contacted at a rate of about 5×10¹³ bacteria per hectare.
13. A kit comprising Paraburkholderia bacteria or the composition of any one of items 6-10, preferably together with instructions regarding the use of the kit.
14. Use of Paraburkholderia bacteria for the preparation of a composition, wherein said composition can be a dried powder or a spraying agent.
15. The use of Paraburkholderia bacteria according to item 14, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.

The present invention is further described by reference to the following non-limiting figures and examples.

### Examples 1 to 7 all Relate to the Same Field Trial in 2021

Example 1 describes the preparation of the Paraburkholderia bacteria to be used in the field trial. Examples 2 to 4 describe the experimental setup and the application of the Paraburkholderia bacteria in the trial. Example 1 was performed in the laboratory, whereas Example 2 describes the preparation of the final bacterial suspension for the application in the field trial. Examples 3 and 4 describe the trial setup and the execution of the field trial. Examples 5 and 6 show the results. Example 7 describes data recording and the analyses of the data obtained in the trial.

### Examples 8 to 15 all Relate to the Same Field Trial in 2022

Example 8 describes the preparation of the Paraburkholderia bacteria to be used in the field trial. Examples 9 to 11 describe the experimental setup and the application of the Paraburkholderia bacteria in the trial. Example 8 was performed in the laboratory, whereas Example 9 describes the preparation of the final bacterial suspension for the application in the field trial. Examples 10 and 11 describe the trial setup and the execution of the field trial. Examples 12 to 14 show the results. Example 15 describes data recording and the analyses of the data obtained in the trial.

### Example 1. Bacterial Strain Preparation

Bacterial strain Paraburkholderia phytofirmans PsJN (accessible in DSMZ-Deutsche Sammlung von Mikroorganismen und Zellkulturen, accession number DSM 17436) was used in the field trial to test the efficacy against the infection of pear (Pyrus communis) with Erwinia amylovora, a causative agent of fire blight. For the preparation of the bacterium P. phytofirmans PsJN, the seed culture was inoculated from the stock of bacterium stored in 20% glycerol at −80° C. It was cultivated in 2 L flasks in a chemically defined liquid medium MM9 containing glucose, ammonium ions, sulfate ions, potassium, magnesium, iron, and calcium) at 28° C. with shaking. The culture was grown until the exhaustion of glucose. The grown culture broth was cooled to 4° C. at the end of the fermentation and stored at 4° C. until use, at most for up to 2 weeks. The number of viable bacteria in the test product has been assessed by performing serial dilutions and colony counting. The number of viable bacteria was consistent during the 2 weeks of storage at 4° C.

### Example 2. Preparation of Spraying Suspension of Bacteria

A spraying suspension was prepared fresh from the culture broth containing P. phytofirmans PsJN on the trial site just before the application to pears. The culture broth was mixed and diluted with tap water to prepare a spraying suspension with concentration of 10⁸ CFU/ml of viable bacteria. The appropriate amount of the adjuvant Break Thru S 301 was added to each of the spraying suspensions and mixed. The concentrations of the adjuvant in the trial were 0.05%, 0.1% and 0.2% (% indicates volume percent) in the final suspension to be applied. The deviation of the intended dose rates per plot was below +/−5%. Break Thru S 301 is a non-ionic spreading and a penetration surfactant (a polyethersiloxane/polyether trisiloxane) that improves the absorption of plant-protecting active ingredients contained within a spraying suspension onto the plant tissue. Once added to the spraying suspension it considerably lowers the surface tension of the water so that the applied drops adhere better to the plant surface.

### Example 3. Treatment Applications

Field experiments were performed to test the efficacy of the bacterial strain P. phytofirmans PsJN to prevent and/or control fire blight caused by Erwinia amylovora on pears (Pyrus communis), common pear variety Santa Maria. The test was performed in a field orchard in Greece, Nisi, in the area of Imathis. The trial location was on a flatland area with dry-warm climate. The trial site was well exposed and was set up in a practice field. The soil preparation followed good agricultural practices and was performed by the local farmer. Crop stand and development were homogeneous across the trial. The pest presence was homogeneous across the trial and at the usual infestation level for the trial area for period of the year in which the trial was conducted. The trees were 13 years old with an average height of 2.8 m and a total canopy height of 2.3 m. The total leaf wall area was 13143 m²/ha. All field experiments included an untreated control, a control treatment (Serenade Max, Bayer, contains Bacillus amyloliquefaciens QST 713), and 3 test treatments with P. phytofirmans PsJN with surfactant Break Thru S 301 at three different concentrations (0.05%, 0.1%, and 0.2%; % denotes volume percent). Application timings for applications to flowers or flowers and/or leaves were during the flowering period with an approx. 10-day interval in between (9^{th} of Apr. 2021, 19^{th} of Apr. 2021 and 27^{th} of Apr. 2021) to maximise colonisation. Treatment A was performed at the beginning of flowering (at BBCH 61). Treatment B was performed at full flowering, when 40-50% flowers on main raceme open, older petals are falling (at BBCH 64-65). Treatment C was performed at decline of flowering with majority of petals fallen (at BBCH 67). The test treatments and the control treatments were conducted with a back-mounted research lance sprayer at 5 bars and 500 liters of water (control) or bacterial suspension (test) per hectare. A suspension of about 10⁸ CFU/mL in water was used for test treatments with P. phytofirmans PsJN which corresponds to 5×10¹³ CFU/ha (5×10¹³ bacteria per hectare) of P. phytofirmans PsJN that was applied. Serenade Max was used according to the instructions of the manufacturer at 4 kg/ha.

### Example 4. Experimental Study Design

The treatments consisted of an untreated control, a treated control (Bacillus amyloliquefaciens QST 713, Serenade Max, Bayer), and three test treatments with P. phytofirmans PsJN with three concentrations of adjuvant Break Thru S 301 (which is a polyethersiloxane/polyether trisiloxane). One replicated small plot trial was carried out. The plants were arranged in a row. Row spacing was 3.5 m, and spacing between trees within a row was 2.5 m. The experiment was designed in Fisher blocks, with four replicates with five trees per elementary plot. Having five trees per elementary plot allowed to make the assessment at the centre of the block and to exclude the trees at the edge of the plot from the assessment to avoid interaction with the neighbouring plots. Four assessments were made during the conduct of the trial, at each trial visit and 10 days after the last application.

### Example 5. Assessment of Crop Safety

The pear trees were observed for phytotoxicity symptoms on leaves. Assessments were done at 0, 10-, 18-, 28-, and 40-days post first application (DAF). One of five plants was assessed. Crop safety (Selectivity) was assessed on an overall plot basis where 0% exhibits no symptoms of phytotoxicity. Scores between 0 and 100 were relative to the untreated plots. The results are shown in Table 2. No phytotoxic symptoms were observed during the assessment timings. Means followed by the same letter (a) do not significantly differ (P=0.05, Student-Newman-Keuls).

TABLE 2
Assessment of phytotoxicity on pears.

| Treatment | | | Application | 10 days post | | 18 days post | | 28 days post | | 40 days post | |
|---|---|---|---|---|---|---|---|---|---|---|---|
| No. | Treatment | Rate | code | 1^{st} appl. | | 1^{st} appl. | | 1^{st} appl. | | 1^{st} appl. | |
| 1 | Untreated Check | | | 0 | a | 0 | a | 0 | a | 0 | a |
| 2 | P. phytofirmans PsJN, | 0.833 | ABC | 0 | a | 0 | a | 0 | a | 0 | a |
| | Break Thru S 301 | I/100 I | | | | | | | | | |
| | 0.05% | | | | | | | | | | |
| 3 | P. phytofirmans PsJN, | 0.833 | ABC | 0 | a | 0 | a | 0 | a | 0 | a |
| | Break Thru S 301 | I/100 I | | | | | | | | | |
| | 0.1% | | | | | | | | | | |
| 4 | P. phytofirmans PsJN, | 0.833 | ABC | 0 | a | 0 | a | 0 | a | 0 | a |
| | Break Thru S 301 | I/100 I | | | | | | | | | |
| | 0.2% | | | | | | | | | | |
| 5 | Serenade Max | 4.0 | ABC | 0 | a | 0 | a | 0 | a | 0 | a |
| | | kg/ha | | | | | | | | | |

| | | | | |
|---|---|---|---|---|
| LSD P = .05 | . | . | . | . |
| Standard Deviation | 0.0 | 0.0 | 0.0 | 0.0 |
| CV | 0.0 | 0.0 | 0.0 | 0.0 |
| Grand Mean | 0.0 | 0.0 | 0.0 | 0.0 |
| Levene's F\hat{ } | . | . | . | . |
| Levene's Prob(F) | . | . | . | . |
| Replicate F | 0.000 | 0.000 | 0.000 | 0.000 |
| Replicate Prob(F) | 1.0000 | 1.0000 | 1.0000 | 1.0000 |
| Treatment F | 0.000 | 0.000 | 0.000 | 0.000 |
| Treatment Prob(F) | 1.0000 | 1.0000 | 1.0000 | 1.0000 |

### Example 6. Assessment of Efficacy on Flowers and Fruit

The efficacy of P. phytofirmans PsJN to prevent E. amylovora infection and disease in the flower clusters of pears was compared to the efficacy of Serenade Max and the untreated control. Four assessments were done by counting the total number of infected flower clusters or shoot tips on each plot at 0, 10-, 18-, and 28-days post first application. The pest severity was assessed visually and counted according to the trial protocol. Assessments were done on flower clusters (n=100) at flowering stages (BBCH 61-69) which were observed for infection with Erwinia amylovora. Efficacy was calculated with the transformation Abbott.

One additional assessment was done on fruits. The assessment was carried out at 22 days post last application (40 days post first application (DAF)) by counting the total number of fruits per plot and the total number of damaged fruits per tree (stage BBCH 71-73). Three (3) trees (n=3) were evaluated and the average determined per 1 tree (n=1). Statistical analysis was done as described in Example 7.

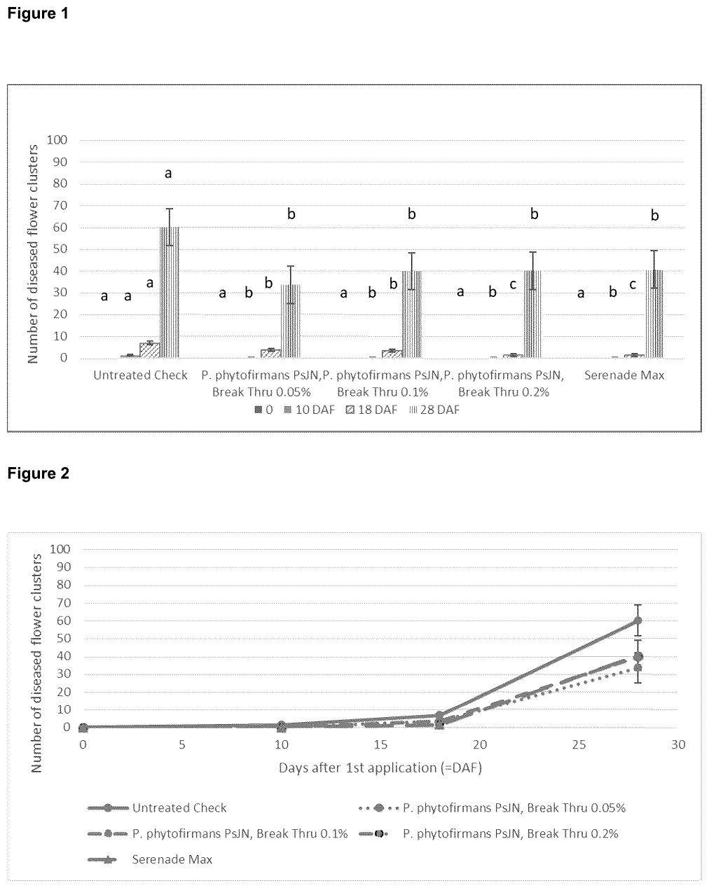

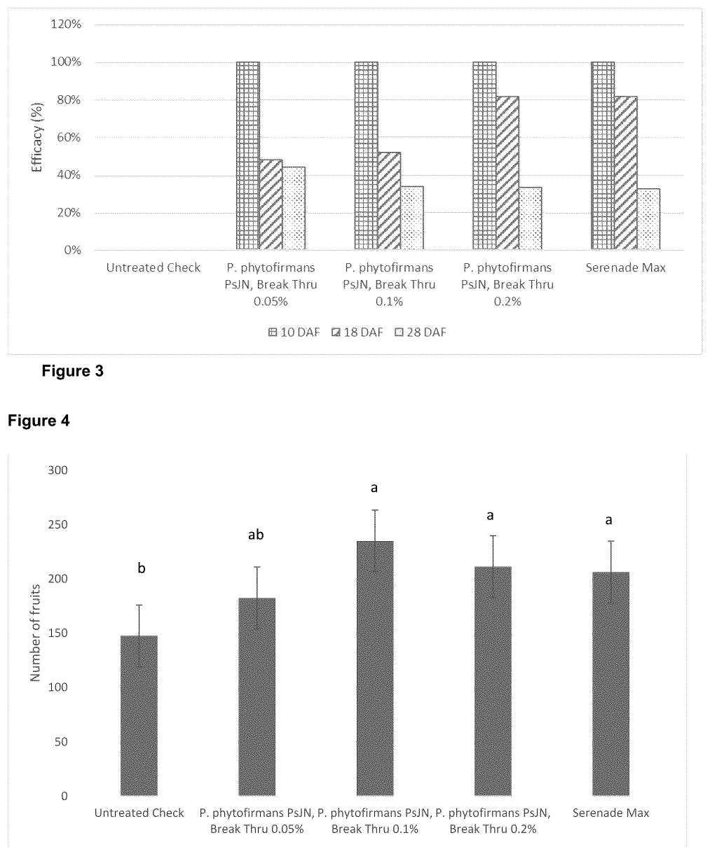

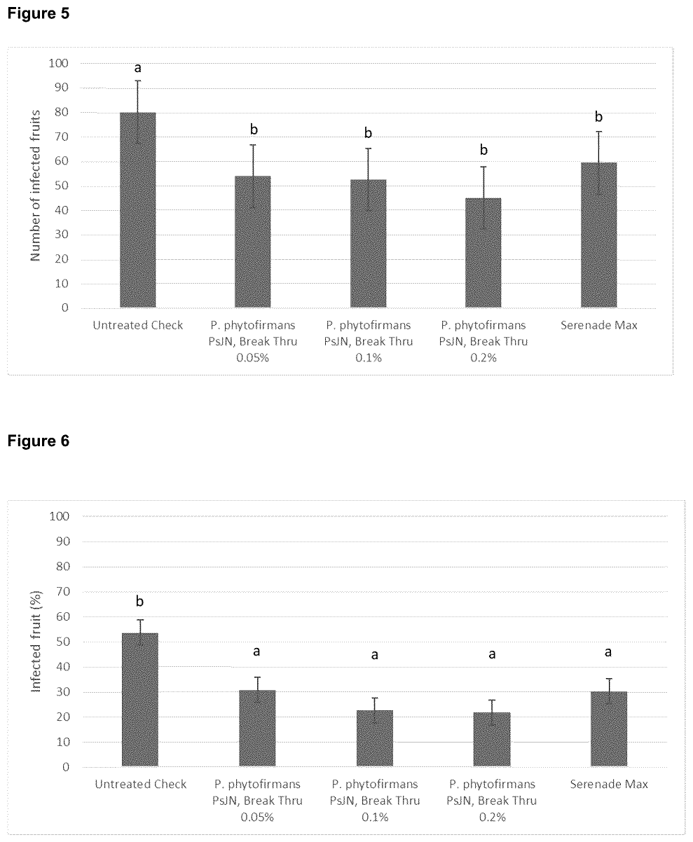

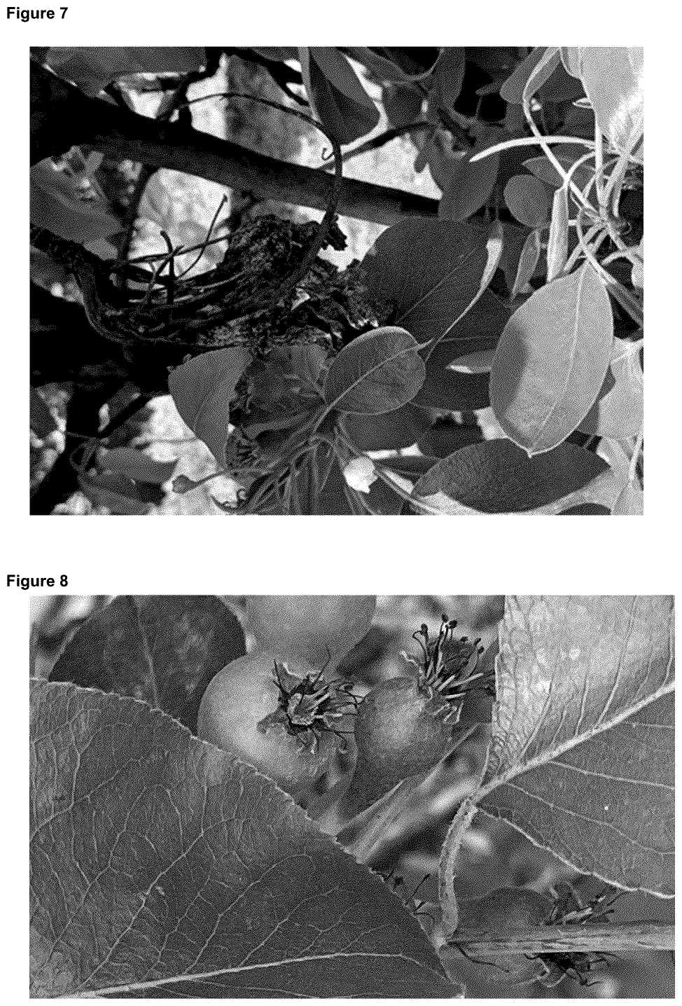

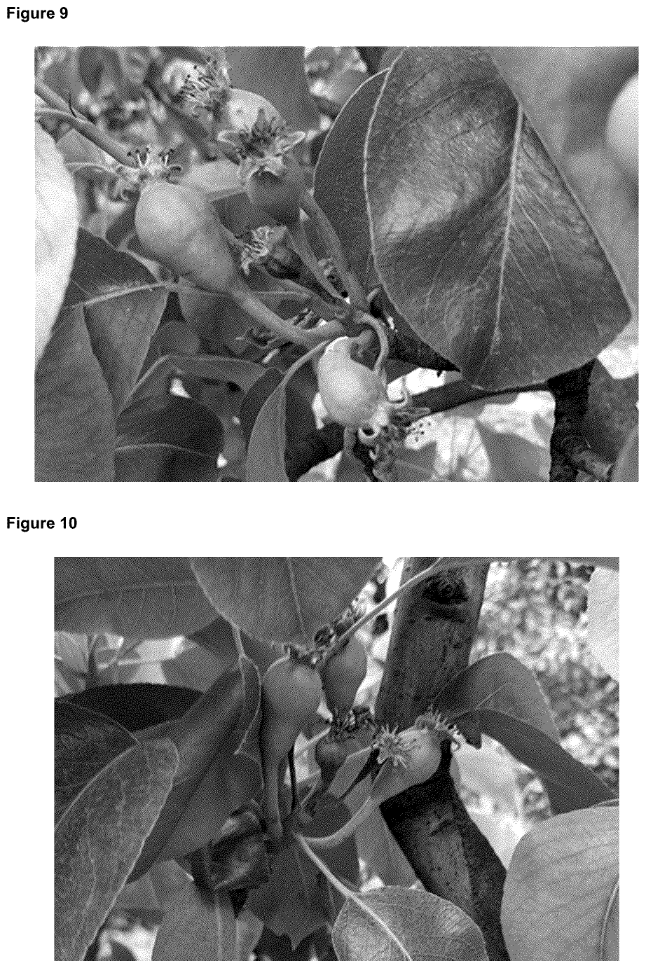

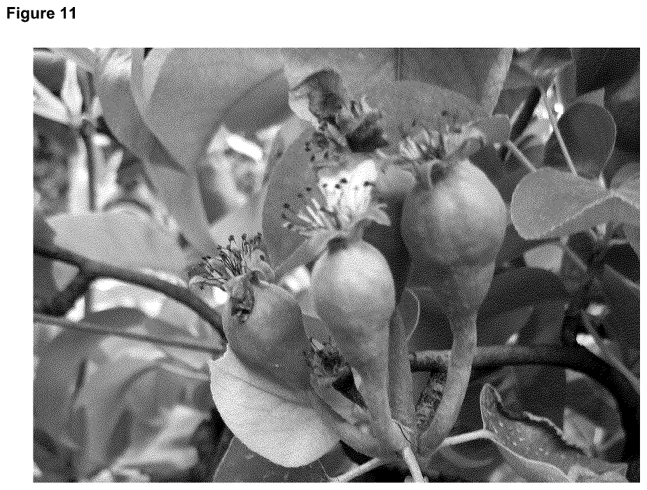

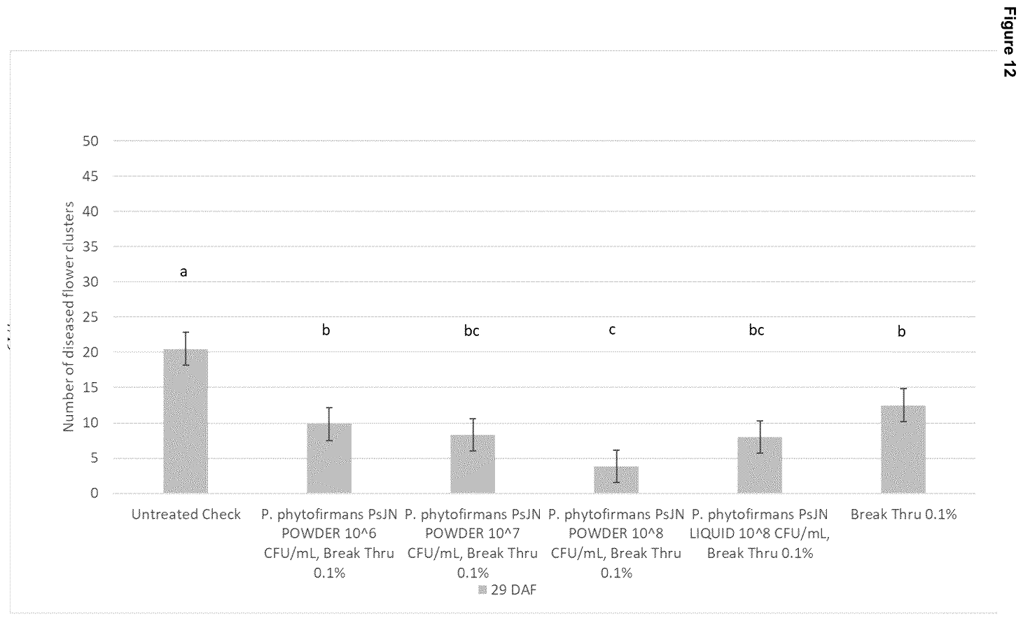

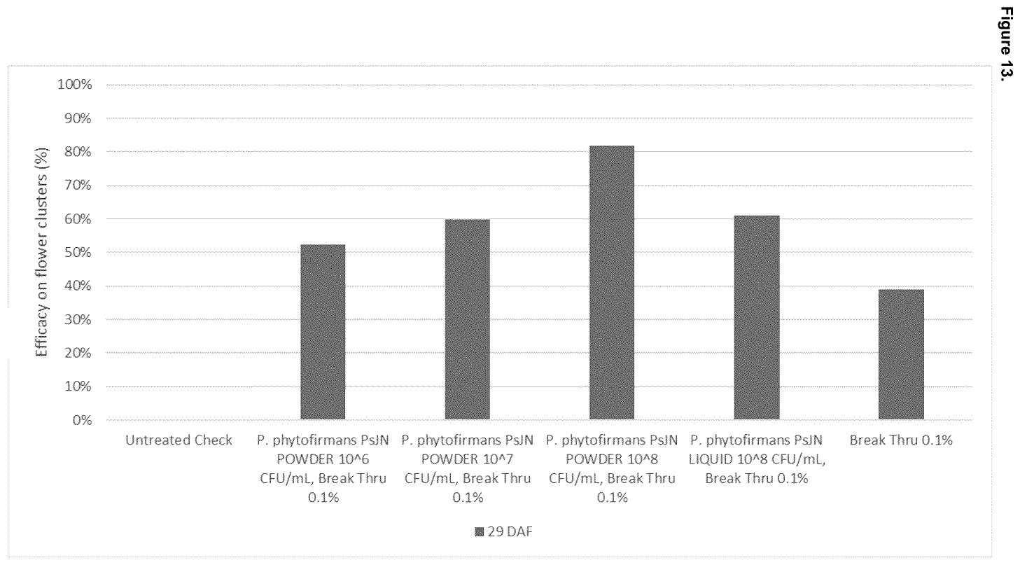

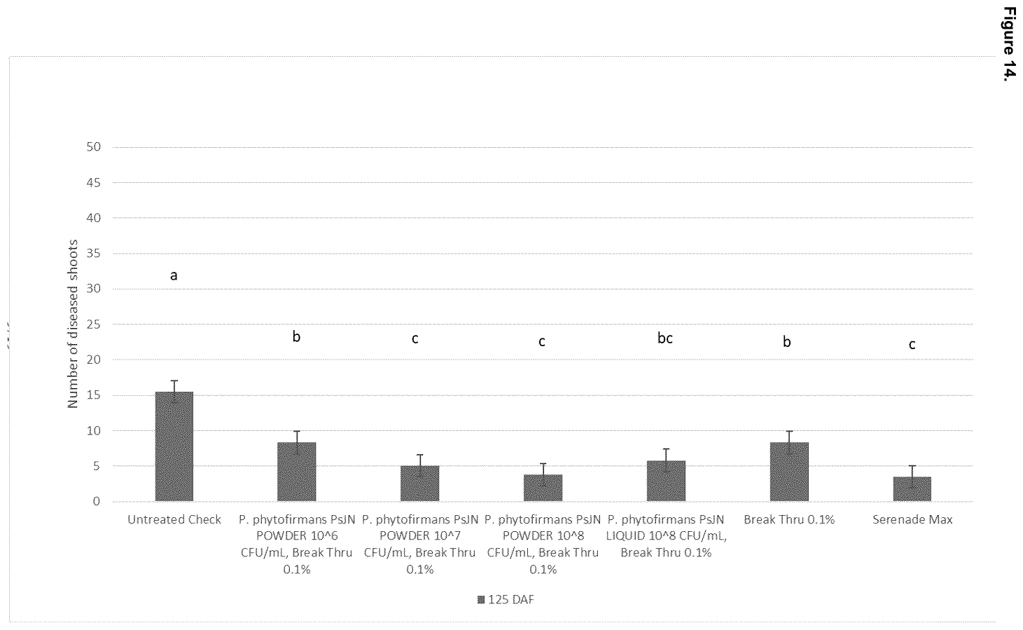

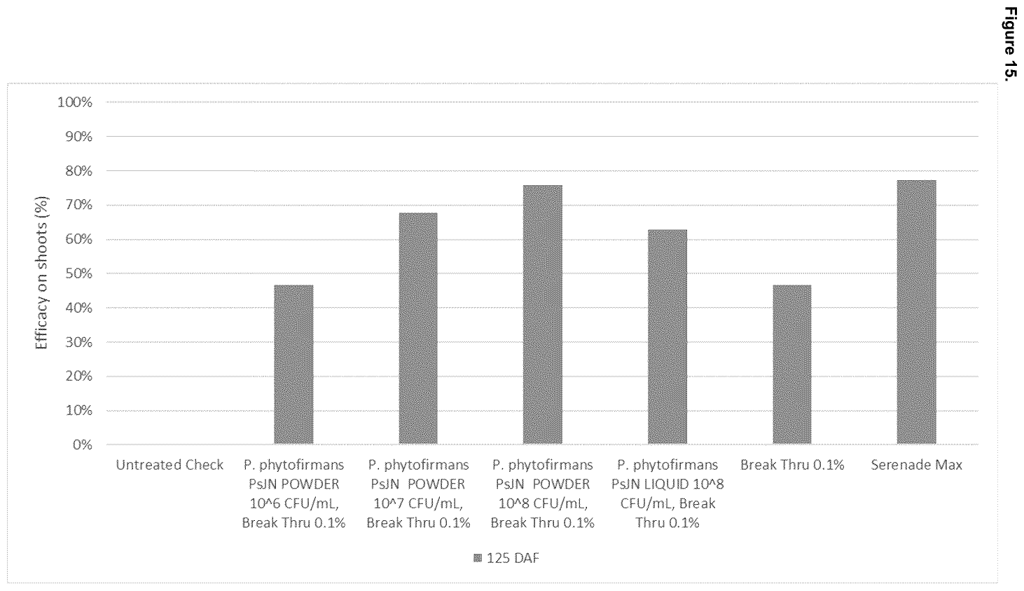

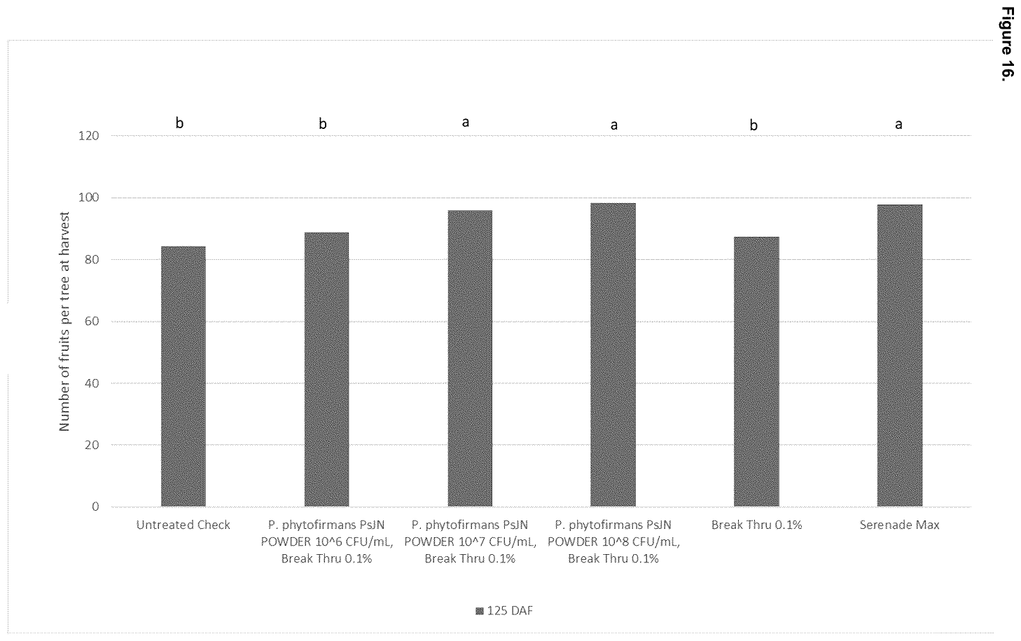

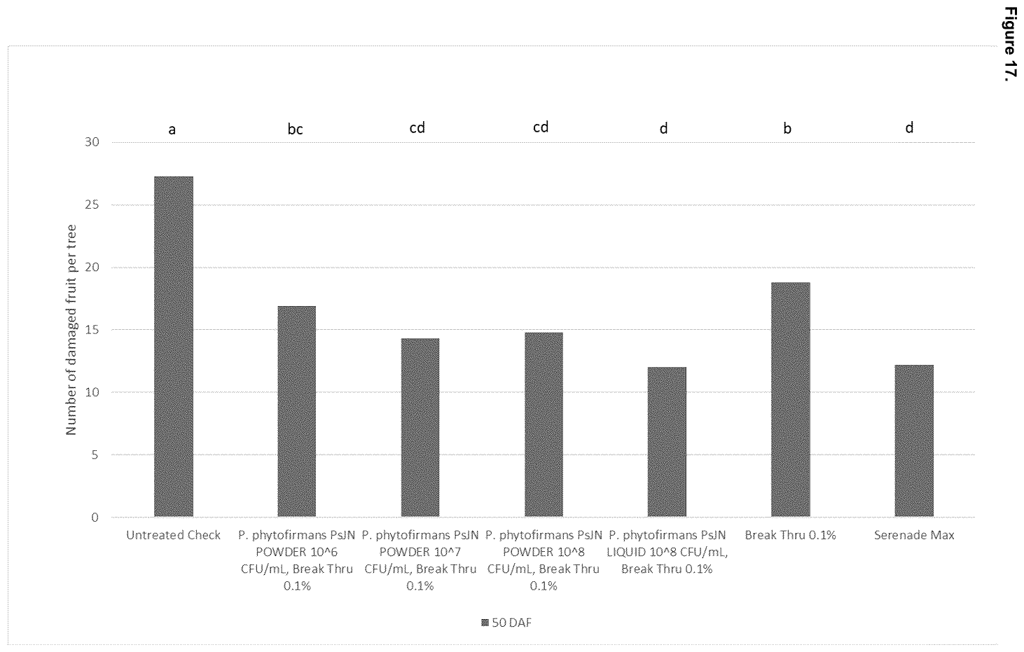

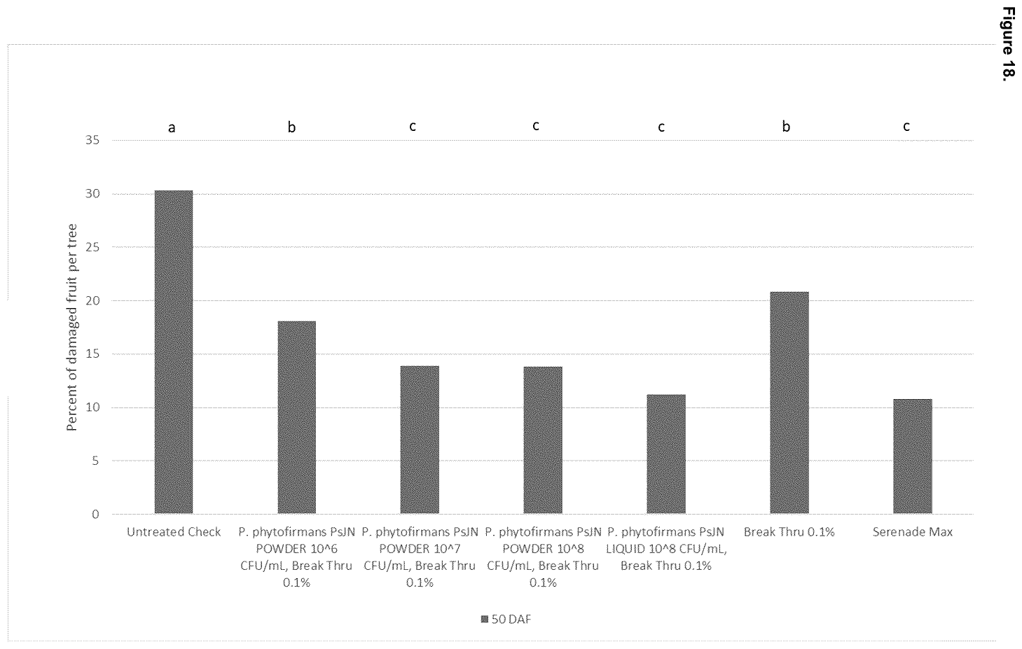

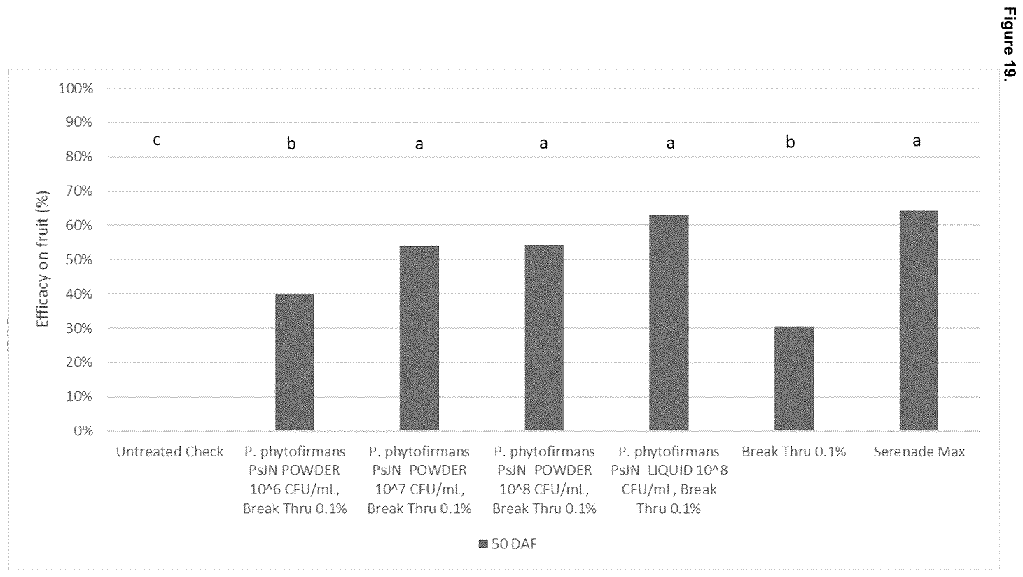

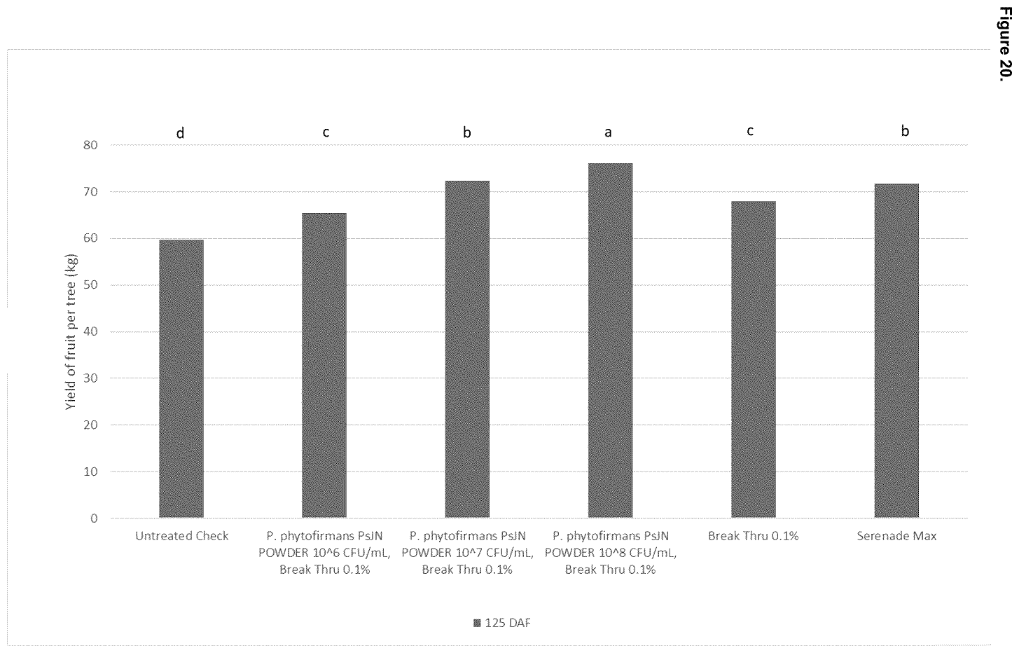

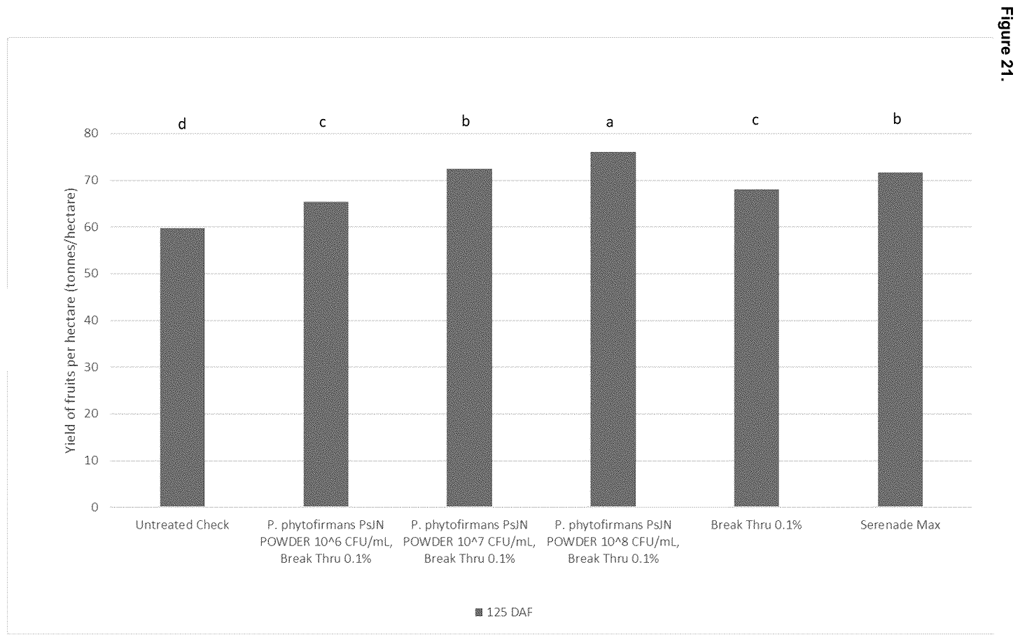

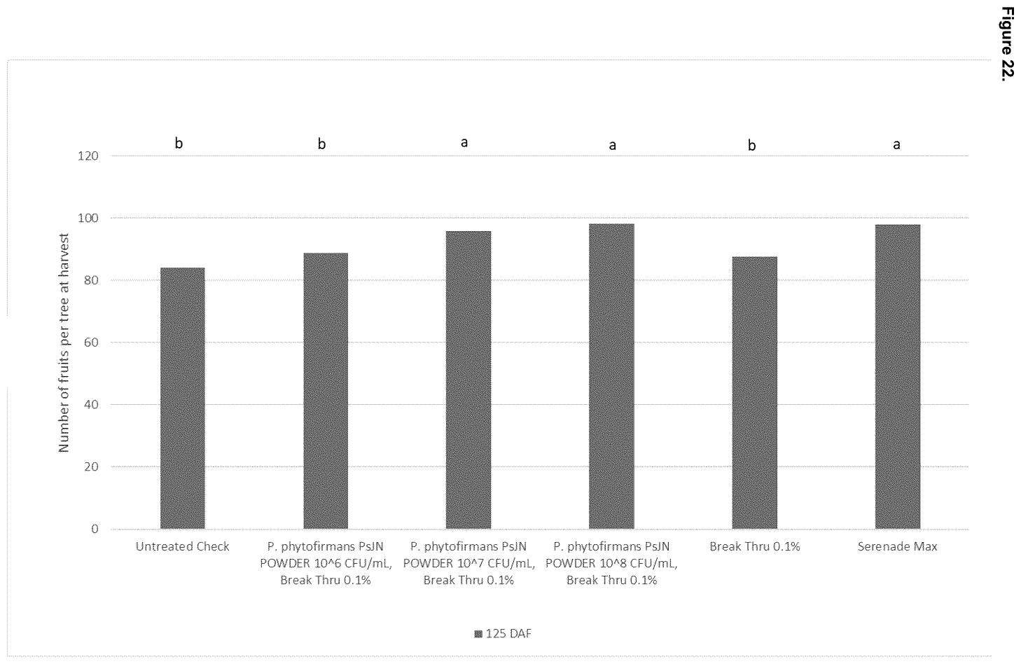

The results are shown in Table 3 and in FIGS. 1 to 6. P. phytofirmans PsJN in tank mix with Break Thru S 301 at all three doses applied (0.05%, 0.1% and 0.2%) controlled Erwinia amylovora on pear.

According to this trial results, all treatments were statistically better compared to the untreated control and similar in efficacy to the reference item Serenade Max in both parameters assessed (i.e. flower clusters (FIGS. 1-3) and fruits (FIGS. 4-6)).

The test item P. phytofirmans PsJN applied in a tank mix with the adjuvant Break Thru S 301 at 0.05% v/v, 0.1% v/v and 0.2% v/v showed similar efficacy compared to Serenade Max (Table 3, FIGS. 1-3 (flower clusters)).

The treatments of P. phytofirmans PsJN applied in tank mix with Break Thru S 301 at 0.1% and 0.2% presented numerically better protection (21.7% and 22.6% respectively) in terms of lower percentages of fruits damaged by Erwinia amylovora, compared to Serenade Max (30.1%), but with no statistically significant difference (Table 3; see FIGS. 4-6 (fruits); data for infected fruits not shown in Table 3). As shown in Table 3 and FIGS. 4-6, all treatments using P. phytofirmans PsJN were statistically better compared to the untreated control and increased efficacy compared to Serenade Max.

TABLE 3
Efficacy of P. phytofirmans PsJN against Erwinia amylovora on flower clusters and fruit.

| Trt. | Treatment | | Appl. | | | 10 days post | | 18 days post | | 28 days post | | 40 days post | |
|---|---|---|---|---|---|---|---|---|---|---|---|---|---|
| No. | name | Rate | Code | 0 | | 1^{st} appl. | | 1^{st} appl. | | 1^{st} appl. | | 1^{st} appl. | |
| | | | | | | Flower clusters (FIG. 1-3) | | | | | | Fruit (FIG. 4) | |
| 1 | Untreated | | | 0 | a | 1.25 | A | 6.75 | a | 60.00 | a | 147.5 | b |
| | Check | | | | | (0.0%) | | (0.0%) | | (0.0%) | | | |
| 2 | P. | 0.833 | ABC | 0 | a | 0.00 | B | 3.50 | b | 33.50 | b | 182.5 | ab |
| | phytofirmans | 1/100 | | | | (100.0%) | | (48.1%) | | (44.2%) | | | |
| | PsJN, Break | 1 | | | | | | | | | | | |
| | Thru S 301 | | | | | | | | | | | | |
| | 0.05% | | | | | | | | | | | | |
| 3 | P. | 0.833 | ABC | 0 | a | 0.00 | B | 3.25 | b | 39.75 | b | 235.0 | a |
| | phytofirmans | 1/100 | | | | (100.0%) | | (51.9%) | | (33.8%) | | | |
| | PsJN, Break | 1 | | | | | | | | | | | |
| | Thru S 301 | | | | | | | | | | | | |
| | 0.1% | | | | | | | | | | | | |
| 4 | P. | 0.833 | ABC | 0 | a | 0.00 | B | 1.25 | c | 40.00 | b | 211.3 | a |
| | phytofirmans | 1/100 | | | | (100.0%) | | (81.5%) | | (33.3%) | | | |
| | PsJN, Break | 1 | | | | | | | | | | | |
| | Thru S 301 | | | | | | | | | | | | |
| | 0.2% | | | | | | | | | | | | |
| 5 | Serenade | 4.0 | ABC | 0 | a | 0.00 | B | 1.25 | c | 40.50 | b | 206.3 | a |
| | Max | kg/ha | | | | (100.0%) | | (81.5%) | | (32.5%) | | | |

| | | | | | |
|---|---|---|---|---|---|
| LSD P =. 05 | . | 0.345 | 1.089 | 13.237 | 43.90 |
| Standard Deviation | 0.0 | 0.224 | 0.707 | 8.592 | 28.49 |
| CV | 0.0 | 89.44 | 22.1 | 20.1 | 14.5 |
| Grand Mean | 0.0 | 0.250 | 3.200 | 42.750 | 196.50 |
| Levene's F\hat{ } | . | 0.45 | 1.804 | 1.282 | 2.278 |
| Levene's Prob(F) | . | 0.771 | 0.181 | 0.321 | 0.109 |
| Rank X2 | . | . | . | . | . |
| P(Rank X2) | . | . | . | . | . |
| Skewness\hat{ } | . | 1.8758* | 0.5437 | −0.3423 | −0.2908 |
| Kurtosis\hat{ } | . | 6.3489* | −0.4413 | 1.7574 | 0.8745 |
| Replicate Prob(F) | 1.0000 | 0.4262 | 0.3096 | 0.0605 | 0.0001 |
| Treatment F | 0.000 | 25.000 | 40.600 | 5.483 | 5.410 |
| Treatment Prob(F) | 1.0000 | 0.0001 | 0.0001 | 0.0095 | 0.0100 |

Legend to Table 3:
The numbers given for flower clusters refer to the total number of infected flower clusters. For example, there is no incidence of disease if the number is 0.
The percentages given refer to the efficacy of treatment with P. phytofirmans against Erwinia amylovora. For example, “100%” means 100% efficacy. The efficacy is calculated based on Abbott's formula, which compares the number of infected flower clusters in treated trees versus the number of infected flower clusters in untreated control.
Numbers followed by the same letter (a, ab, b or c, respectively) do not significantly differ from each other (P = .05, Student-Newman-Keuls). The treatments at the same time of evaluation are compared (i.e. at 0, 18, 28 DAF, see the respective results in the columns).

### Example 7. Trial Data and Statistical Analysis

Trial management, data capture, and statistical analysis have been done with Agriculture Research Manager (ARM) software (developed by Gylling Data Management, Inc.). Results were analysed using Arable Research Manager (ARM) to give analysis of variance and separation of the treatment means using the Student-Newman-Keuls multiple range test. Assessment data were analysed using a two-way analysis of variance (ANOVA) on untransformed and transformed data. The probability of no significant differences occurring between treatment means is calculated as the F probability value (p (F)). Significant differences implied between means where the p (F) value is greater than 0.05 derived as correspondingly lower levels than the generally accepted 95% confidence limit. A letter test was then applied to separate any treatment differences that may be implied by the ANOVA test (Prob (F)<0.05), indicated by the LSD-value and by the letter-test.

Agriculture Research Manager (ARM) must estimate values for missing data points since the data analysis techniques require balanced data within treatments. The method used was YATES.

### Example 8. Bacterial Strain Preparation

The culture for the 2022 field trial was prepared as described in Example 1. The grown culture broth was cooled to 4° C. at the end of the fermentation and stored at 4° C. until use, at most for up to 2 weeks (liquid).

The dried formulation used as powder was prepared from biomass obtained by centrifugation, which was mixed with cryoprotectants, frozen at −80° C., and freeze-dried. Several additives can be used as cryoprotectants, such as saccharose, lactose, trehalose, skimmed milk or similar. The number of viable bacteria in the test product has been assessed by performing serial dilutions and colony counting. The number of viable bacteria was consistent during 2 weeks of storage at 4° C.

The preparation from Example 1 was used for the liquid test product, i.e., bacteria directly derived from liquid culture/culture broth without freeze-drying and without preparation of powder (“LIQUID” in the figures). By contrast, dry bacteria were obtained by freeze-drying which was then used for the preparation of a suspension for application in the field trial (“POWDER” in the figures).

### Example 9. Preparation of Spraying Suspension of Bacteria

A spraying suspension for field trial in 2022 was prepared fresh from the culture broth and the dried biomass on the trial site just before the application to pears as described in Example 2. The culture broth containing P. phytofirmans PsJN (liquid) was mixed and diluted with tap water to prepare a spraying suspension with concentration of 10⁸ CFU/ml of viable bacteria. The test items containing dried P. phytofirmans PsJN (powder) were mixed and diluted with tap water to prepare a spraying suspension with a concentration of 10⁶, 10⁷ or 10⁸ CFU/ml of viable bacteria. The appropriate amount of the adjuvant Break Thru S 301 was added to each of the spraying suspensions and mixed. The concentration of the adjuvant in the trial was 0.1% (% indicates volume percent) in the final suspension to be applied. Based on the data in the 2021 trial, 0.1% Break Thru S 301 was selected for the 2022 trial.

The deviation of the intended dose rates per plot was below +/−5%.

### Example 10. Treatment Applications

Second field experiment was performed in 2022 to test the efficacy of the bacterial strain P. phytofirmans PsJN to prevent and/or control fire blight caused by Erwinia amylovora on pears (Pyrus communis), common pear variety Santa Maria. The test was performed in a field orchard in Greece, Nisi, in Imathis, in the same location as the first trial in 2021. The trial location was in a flatland area with a dry-warm climate. The trial site was well-exposed and was set up in a practice field. The soil preparation followed good agricultural practices and was performed by the local farmer. Crop stand and development were homogeneous across the trial. The pest presence was homogeneous across the trial and at the usual infestation level for the trial area for the year in which the trial was conducted. In general, the weather conditions were normal for the trial area, but the humidity which is a major factor for the Erwinia amylovora infestation level was lower than the previous year. The trees were 13 years old with an average height of 2.5 m at the first application and 3.2 m at the last application, and a total canopy height of 2.1 m at the first application and 2.7 m at the last application. The total leaf wall area was 12000 m²/ha at the first application and 15429 m²/ha at the last application.

All field experiments included an untreated control (untreated check), a control treatment (Serenade Max, Bayer, contains Bacillus amyloliquefaciens QST 713), a control treatment with surfactant Break Thru S 301 at 0.1% (% denotes volume percent), and four test treatments with P. phytofirmans PsJN with surfactant Break Thru S 301 at 0.1% (% denotes volume percent) and with different state of P. phytofirmans PsJN (liquid versus powder). The liquid formulation was tested at 10⁸ CFU/ml, whereas powder was tested at 10⁶, 10⁷ and 10⁸ CFU/ml. Application timings for applications to flowers or flowers and/or leaves were during the flowering period (12^{th} of Apr. 2022, 21^{st} of Apr. 2022 and 28^{th} of Apr. 2022). Treatment A was performed at the beginning of flowering (at BBCH 61). Treatment B was performed at full flowering, when 40-50% of flowers on the main raceme open, and older petals are falling (at BBCH 64-65). Treatment C was performed at the decline of flowering with a majority of petals fallen (at BBCH 67). The pest stage at each application was pre-infection. The test treatments and the control treatments were conducted with a back-mounted research lance sprayer at 8 bars and 600 litres of water (control) or bacterial suspension (test) per hectare in the first application, and 800 litres of water (control) or bacterial suspension (test) per hectare in the second and third application. A suspension of about 10⁶, 10⁷ or 10⁸ CFU/mL in water was used for test treatments with P. phytofirmans PsJN which corresponds to 5×10¹¹, 5×10¹², or 5×10¹³ CFU/ha (5×10¹¹, 5×10¹², or 5×10¹³ bacteria per hectare) of P. phytofirmans PsJN that was applied. Serenade Max was used according to the instructions of the manufacturer at 4 kg/ha.

### Example 11. Experimental Study Design

The treatments in the 2022 field trial consisted of untreated control, a treated control (Bacillus amyloliquefaciens, Serenade Max, Bayer), a test treatment with surfactant Break Thru S 301 at 0.1% (% denotes volume percent), and four test treatments, of which three test treatments with freeze-dried P. phytofirmans PsJN in different cell concentrations (10⁶, 10⁷ or 10⁸ CFU/mL) and with surfactant Break Thru S 301 (which is a polyethersiloxane/polyether trisiloxane) at 0.1% (% denotes volume percent), and a test treatment with P. phytofirmans PsJN in liquid at 10⁸ CFU/ml and with surfactant adjuvant Break Thru S 301 at 0.1% (% denotes volume percent). One replicated small plot trial was carried out. The plants were arranged in a row. Row spacing was 3.5 m, and spacing between trees within a row was 2.5 m. As before, the experiment was designed in Fisher blocks, with four replicates with five trees per elementary plot.

Having five trees per elementary plot allowed to make the assessment at the centre of the block and to exclude the trees at the edge of the plot from the assessment to avoid interaction with the neighbouring plots.

Six assessments were made during the conduct of the 2022 trial. From the start of the trial and before each of the three applications, at 13 days after the last application and 34 days after the last application, the presence of phytotoxicity and the infection on flower clusters and on shoots were assessed. At 34 days after the last application, the total number of fruits per tree and the number of damaged fruits were counted. At the crop harvest time on the 15^{th} of August, the total number of fruits per tree was counted and the weight of fruits per plot was measured. The yield per plot was calculated on 3 central trees of each plot. The crop was also assessed visually for the presence of any other symptoms.

### Example 12. Assessment of Crop Safety

In the 2022 trial, the pear trees were observed for phytotoxicity symptoms on leaves. Assessments were done at 0, 16-, 29-, 50-, and 125-days after the first application. Three of five plants were assessed. Crop safety (selectivity) was assessed on an overall plot basis where 0% exhibits no symptoms of phytotoxicity. Scores between 0 and 100 were relative to the untreated plots. No phytotoxic symptoms were observed during the assessment timings by any of the treatments in the 2022 trial.

### Example 13. Assessment of Efficacy on Flowers and Shoots

In the 2022 field trial, the efficacy of P. phytofirmans PsJN to prevent E. amylovora infection and disease in the flower clusters of pears was compared to the efficacy of Serenade Max and the untreated control. Four assessments were done by counting the total number of infected flower clusters on 1 plot at 0, 9, 16, and 29 days after the first application (DAF). The pest severity was assessed visually and counted according to the trial protocol. Assessments were done on flower clusters (n=100) at flowering stages (BBCH 61-69), which were observed for infection with Erwinia amylovora. Efficacy was calculated with the transformation Abbott.

One additional assessment was done on shoots at harvest. The assessment was carried out at 10⁹ days after the last application (125 days after the first application) by counting the total number of damaged shoots in 1 plot based on 3 trees (stage BBCH 87). Statistical analysis was done as described in Example 15.

The first symptoms were observed 13 days after the last application (29 days after the first application (DAF). All treatments of P. phytofirmans PsJN applied in a tank mix with Break Thru S 301 at 0.1% v/v were statistically better at reducing the number of diseased flower clusters compared to the untreated control. The results of efficacy of the treatments on flower clusters are shown in Table 4 and in FIGS. 12 and 13. The highest efficacy on flower clusters between the treatments was observed by P. phytofirmans PsJN powder at 10⁸ CFU/mL (81.7%) at 29 days after the first application. This treatment was statistically similar in efficacy to Serenade Max.

At the last assessment on shoots, at harvest, all P. phytofirmans PsJN treatments were statically better at reducing the number of diseased shoots compared to the untreated control. The results of efficacy of treatments on shoots are shown in Table 5 and in FIGS. 14 and 15. The highest efficacy on shoots among the test treatments with P. phytofirmans PsJN was observed by P. phytofirmans PsJN powder at 10⁸ CFU/mL (75.8%). This treatment was statistically similar in efficacy as P. phytofirmans PsJN powder at 10⁷ CFU/mL, P. phytofirmans PsJN liquid at 10⁸ CFU/mL and Serenade Max.

TABLE 4
Efficacy of P. phytofirmans PsJN against Erwinia amylovora
on flower clusters. Number of diseased flower clusters out of
100 evaluated and efficacy (%) was evaluated at 0, 9, 16, and
29 post first application (DAF). Percentages represent efficacies
based on the comparison of each treatment to untreated control.
Efficacy was calculated with the transformation Abbott. Different
letters (a, b, c) indicate significantly different results.

| Trt | | Appl | Days After First Appl. | | | |
|---|---|---|---|---|---|---|
| No. | Treatment Name | Code | 0 | 9 | 16 | 29 |
| 1 | Untreated Check | | 0a | 0a | (a | 20.5a |
| 2 | P. phytofirmans | ABC | 0a | 0a | (a | 9.8b |
| | PsJN powder | | | | | (52.4%) |
| | 10\hat{ }6 CFU/mL, | | | | | |
| | Break Thru | | | | | |
| | 0.1% v/v | | | | | |
| 3 | P. phytofirmans | ABC | 0a | 0a | (a | 8.3bc |
| | PsJN powder | | | | | (59.8%) |
| | 10\hat{ }7 CFU/mL | | | | | |
| | Break Thru | | | | | |
| | 0.1% v/v | | | | | |
| 4 | Dried | ABC | 0a | 0a | (a | 3.8c |
| | P. phytofirmans | | | | | (81.7%) |
| | PsJN powder | | | | | |
| | 10\hat{ }8 CFU/mL | | | | | |
| | Break Thru | | | | | |
| | 0.1% v/v | | | | | |
| 5 | P. phytofirmans | ABC | 0a | 0a | (a | 8.0bc |
| | PsJN powder | | | | | (61.0%) |
| | 10\hat{ }8 CFU/mL | | | | | |
| | Break Thru | | | | | |
| | 0.1% v/v | | | | | |
| 6 | Break Thru | ABC | 0a | 0a | (a | 12.5b |
| | 0.1% v/v | | | | | (39.0%) |

TABLE 5
Efficacy of P. phytofirmans PsJN against Erwinia amylovora
on shoots at fruit harvest (125 days after the first application).
Efficacy was calculated with the transformation Abbott. Different
letters indicate significantly different results.

| | | | Efficacy |
|---|---|---|---|
| | | | on shoots |
| | | | Days After |
| Trt | | Appl | First Appl. |
| No. | Treatment Name | Code | 125 |
| 1 | Untreated Control | ABC | 15.5a |
| | | | (0.0%) |
| 2 | P. phytofirmans PsJN | ABC | 8.3b |
| | powder 10\hat{ }6 CFU/mL, | | (46.8%) |
| | Break Thru 0.1% v/v | | |
| 3 | P. phytofirmans PsJN | ABC | 5.0c |
| | powder 10\hat{ }7 CFU/mL, | | (67.7%) |
| | Break Thru 0.1% v/v | | |
| 4 | P. phytofirmans PsJN | ABC | 3.8c |
| | powder 10\hat{ }8 CFU/mL, | | (75.8%) |
| | Break Thru 0.1% v/v | | |
| 5 | P. phytofirmans PsJN | ABC | 5.8bc |
| | liquid 10\hat{ }8 CFU/mL, | | (62.9%) |
| | Break Thru 0.1% v/v | | |
| 6 | Break-Thru 0.1% v/v | ABC | 8.3b |
| | | | (46.8%) |
| 7 | Serenade Max | ABC | 3.5c |
| | | | (77.4%) |

### Example 14. Assessment of Efficacy on Fruits

In the 2022 field trial, the efficacy of P. phytofirmans PsJN to prevent E. amylovora infection of pears was compared to the efficacy of Serenade Max and the untreated control on fruits.

### Number of Fruits Per Tree:

At the assessment at harvest, 125 days after the first treatment, all treatments numerically increased the number of fruits compared to the untreated control, but statistically significant differences on the number of fruits were determined only with the treatments P. phytofirmans PsJN powder 10⁷ CFU/mL and 10⁸ CFU/mL, and with Serenade Max. The highest number of fruits was produced by the trees treated with P. phytofirmans PsJN powder 10⁸ CFU/mL. The results of efficacy of treatments on the number of fruits are shown in Table 6 and in FIG. 16.

TABLE 6
Efficacy of P. phytofirmans PsJN against Erwinia amylovora
on number of fruits per tree at 50 days post first application
and at harvest (125 days after the first application). Efficacy
(%) was calculated with the transformation Abbott. Different
letters (a, b) indicate significantly different results.

| | | | Number of healthy |
|---|---|---|---|
| | | | fruits per tree |
| | | | Days After |
| Trt | | Appl | First Applic. |
| No. | Treatment Name | Code | 125 |
| 1 | Untreated Control | | 84.2b |
| 2 | P. phytofirmans PsJN | ABC | 88.8b |
| | POWDER 10\hat{ }6 CFU/mL, | | |
| | Break Thru 0.1% v/v | | |
| 3 | P. phytofirmans PsJN | ABC | 95.8a |
| | POWDER 10\hat{ }7 CFU/mL, | | |
| | Break Thru 0.1% v/v | | |
| 4 | P. phytofirmans PsJN | ABC | 98.3a |
| | POWDER 10\hat{ }8 CFU/mL, | | |
| | Break Thru 0.1% v/v | | |
| 6 | Break-Thru 0.1% v/v | | 87.5b |
| 7 | Serenade Max | | 97.9a |

### Damaged Fruits:

At the assessment timing 50 days after the first treatment, all treatments decreased the percentage of damaged fruits compared to the untreated control, with a statistically significant difference.

P. phytofirmans PsJN liquid 10⁸ CFU/mL presented numerically the best result between the P. phytofirmans PsJN test treatments. P. phytofirmans PsJN liquid 10⁸ CFU/mL was statistically similar as the treatments with P. phytofirmans PsJN powder 10⁷ CFU/mL and 10⁸ CFU/mL, and Serenade Max. The results are shown in Table 7 and in FIGS. 17, 18, and 19.

TABLE 7
Efficacy of P. phytofirmans PsJN against Erwinia amylovora,
number and percentage of damaged fruits per tree at 50 days
after the first application. Efficacy (%) was calculated
with the transformation Abbott. Different letters (a, b,
c) indicate significantly different results.

| | | | Number of | % of | |
|---|---|---|---|---|---|
| | | | damaged | damaged | |
| | | | fruits per | fruits per | Efficacy |
| | | | tree | tree | (%) |
| Trt | | Appl | Days After First Applic. | | |
| No. | Treatment Name | Code | 50 | 50 | 50 |
| 1 | Untreated Check | | 27.3a | 30.3a | 0.0c |
| 2 | ACIES AB POWDER | ABC | 16.9bc | 18.1b | 39.8b |
| | 10\hat{ }6 CFU/mL | | | | |
| | Break-Thru | | | | |
| 3 | ACIES AB POWDER | ABC | 14.3cd | 13.9c | 53.9a |
| | 10\hat{ }7 CFU/mL | | | | |
| | Break-Thru | | | | |
| 4 | ACIES AB POWDER | ABC | 14.8cd | 13.8c | 54.3a |
| | 10\hat{ }8 CFU/mL | | | | |
| | Break-Thru | | | | |
| 5 | ACIES AB LIQUID | ABC | 12.0d | 11.2c | 63.0a |
| | 10\hat{ }8 CFU/mL | | | | |
| | Break-Thru | | | | |
| 6 | Break-Thru | ABC | 18.8b | 20.8b | 30.6b |
| 7 | Serenade Max | ABC | 12.2d | 10.8c | 64.3a |

### Yield of Fruits:

At harvest, 125 days after the first treatment, all P. phytofirmans PsJN treatments statistically increased the yield of fruits compared to the untreated control. In the treatment with P. phytofirmans PsJN powder 10⁸ CFU/mL, the production was the highest between the trial treatments and statistically superior compared to all other trial treatments. The production in the treatment with P. phytofirmans PsJN powder 10⁷ CFU/mL was similar to the production under treatment with Serenade Max. The yields of fruit at harvest in tonnes per hectare and in kilograms per tree are shown in Table 8 and in FIGS. 20 and 21.

TABLE 8
Yield of fruit at harvest (125 days after the first application).
Different letters indicate significantly different results.

| | | | Yield per | Yield per tonnes |
|---|---|---|---|---|
| | | | tree (kg) | per hectare |
| Trt | | Appl | Days After First Applic. | |
| No. | Treatment Name | Code | 125 | 125 |
| 1 | Untreated Control | | 59.70d | 22.7d |
| 2 | P. phytofirmans PsJN | ABC | 65.40c | 24.9c |
| | POWDER 10\hat{ }6 CFU/mL, | | | |
| | Break Thru 0.1% v/v | | | |
| 3 | P. phytofirmans PsJN | ABC | 72.40b | 27.6b |
| | POWDER 10\hat{ }7 CFU/mL, | | | |
| | Break Thru 0.1% v/v | | | |
| 4 | P. phytofirmans PsJN | ABC | 76.10a | 29.0a |
| | POWDER 10\hat{ }8 CFU/mL, | | | |
| | Break Thru 0.1% v/v | | | |
| 6 | Break-Thru 0.1% v/v | ABC | 68.00c | 25.9c |
| 7 | Serenade Max | ABC | 71.70b | 27.3b |

### Weight Per Fruit:

The weight of fruit in each of the treatments was assessed at harvest, 125 days after the first treatment. All P. phytofirmans PsJN treatments numerically increased the weight per fruit compared to the untreated control and Serenade Max. The weight of the fruit obtained from the trees treated with P. phytofirmans PsJN powder 10⁸ CFU/mL was the highest between the test treatments, but no statistically significant difference existed between the trial treatments. In addition, the number of fruits per 3 trees was also the highest in the trees treated with P. phytofirmans PsJN powder 10⁸ CFU/mL. The results are shown in Table 9 and FIG. 22.

TABLE 9
Efficacy of P. phytofirmans PsJN against
Erwinia amylovora - Weight per fruit in
kilograms at harvest. Different letters
indicate significantly different results.

| | | | Number of | Weight of 1 |
|---|---|---|---|---|
| | | | fruits per | fruit (in kg), |
| | | | 3 trees | per 3 trees |
| Trt | | Appl | Days After First Applic. | |
| No. | Treatment Name | Code | 125 | 125 |
| 1 | Untreated Control | | 252.5b | 0.237a |
| 2 | P. phytofirmans PsJN | ABC | 266.3b | 0.246a |
| | POWDER 10\hat{ }6 CFU/mL, | | | |
| | Break Thru 0.1% v/v | | | |
| 3 | P. phytofirmans PsJN | ABC | 287.5a | 0.252a |
| | POWDER 10\hat{ }7 CFU/mL, | | | |
| | Break Thru 0.1% v/v | | | |
| 4 | P. phytofirmans PsJN | ABC | 295.0a | 0.258a |
| | POWDER 10\hat{ }8 CFU/mL, | | | |
| | Break Thru 0.1% v/v | | | |
| 6 | Break-Thru 0.1% v/v | ABC | 262.5b | 0.259a |
| 7 | Serenade Max | ABC | 293.8a | 0.244a |

### Example 15. Trial Data and Statistical Analysis

The 2022 trial management, data capture, and statistical analysis have been done with Agriculture Research Manager (ARM) software (developed by Gylling Data Management, Inc.). Results were analysed using ARM. Assessment data were analysed using a one-way analysis of variance (ANOVA) on untransformed data. The probability of no significant differences occurring between treatment means was calculated as the F probability value pF=0.05 (95% confidence limit). Student-Newman & Keuls' test was then applied to assess any treatment differences identified based on the ANOVA test. The results obtained are indicated by a letter. Treatment means with no letter in common are significantly different in accordance with a Student-Newman and Keuls' test conducted at a 95% confidence level. The ANOVA assumption of homogeneity of variance has been checked using a Levene's chi-square test.

### Example 16. Testing of Antibacterial and Antifungal Activity of Paraburkholderia phytofirmans

P. phytofirmans PsJN was tested for antimicrobial activity against several bacterial and fungal species. Bacillus amyloliquefaciens QST 713 from Serenade Max was tested for its antifungal activity. P. phytofirmans PsJN and B. amyloliquefaciens from Serenade Max was inoculated from the stock stored in 20% glycerol at −80° C. to solid 2TY medium and incubated at 28° C. and at 30° C., respectively. The test bacterial species E. coli and Bacillus subtilis were cultured in liquid 2TY medium at 37° C. with shaking overnight. One hundred millilitres of bacterial culture were spread evenly on the solid 2TY medium, then P. phytofirmans PsJN was swab-inoculated in two parallel lines and the plates were incubated at 28° C. The results of the test are in FIG. 23. The fungal species Aspergilllus niger, Fusarium, Alternaria, Trichoderma, and Botrytis were cultured on PDA solid medium at 25° C. A small part of the mycelium from the plate was transferred to the centre of a plate with solid PDA medium, then P. phytofirmans PsJN or B. amyloliquefaciens QST 713 from Serenade Max were swab-inoculated in two parallel lines on each side of the fungal inoculum and the plates were incubated at 25° C.

P. phytofirmans PsJN did not have any antimicrobial activity against the tested bacterial and fungal species E. coli, and Bacillus subtilis (FIG. 23, bottom row) nor against the tested fungal species Aspergillus niger, Botrytis, Alternaria, Fusarium and Trichoderma (FIG. 24, bottom row). B. amyloliquefaciens QST 713 from Serenade Max had an antifungal effect against the tested fungi. The results are shown in FIG. 24.

1. Paraburkholderia bacteria for use in preventing and/or controlling pests on a plant and/or on a temporary part of a plant and/or being located inside of a plant.

2. Paraburkholderia bacteria for use according to claim 1, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.

3. Paraburkholderia bacteria for use according to claims 1 and 2, wherein said preventing and/or controlling of pests increases undamaged fruit productivity of said plant.

4. Paraburkholderia bacteria for use according to any one of claims 1 to 3, wherein said pests comprise Erwinia amylovora.

5. Paraburkholderia bacteria for use according to any one of claims 1 to 4, wherein said plant belongs to the Rosaceae family of plants.

6. Paraburkholderia bacteria for use according to claim 5, wherein said Rosaceae family of plants comprises pears, peaches, apples, quinces, apricots, plums, cherries, raspberries, loquats, strawberries, rose hips, hawthorns, almonds and roses, preferably wherein said Rosaceae family of plants comprises pears, peaches and apples.

7. Paraburkholderia bacteria for use according to any one of claims 1 to 6, wherein said temporary part of said plant is selected from the group consisting of flowers, leaves, and fruits.

8. Paraburkholderia bacteria for use according to any one of claims 1 to 7, wherein said Paraburkholderia bacteria are formulated in a composition, preferably wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.

9. Paraburkholderia bacteria for use according to claim 8, wherein said composition further comprises at least a second agent and optionally secreted metabolites of said Paraburkholderia bacteria.

10. Paraburkholderia bacteria for use according to claim 9, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores preferably wherein said at least second agent is a surfactant.

11. Paraburkholderia bacteria for use according to claim 10, wherein said surfactant is a polyethersiloxane/polyether trisiloxane.

12. Paraburkholderia bacteria for use according to claims 10 and 11, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.

13. Paraburkholderia bacteria for use according to any one of claims 1 to 12, wherein said Paraburkholderia bacteria or said composition comprising said Paraburkholderia bacteria is contacted to said plant and/or said temporary part of said plant, preferably wherein said Paraburkholderia bacteria or said composition comprising said Paraburkholderia bacteria is contacted to the flowers of said plant.

14. Paraburkholderia bacteria for use according to claim 13, wherein said contacting is performed at least once, at least twice, at least three times, at least four times or at least five times.

15. Paraburkholderia bacteria for use according to claims 13 and 14, wherein said Paraburkholderia bacteria are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein said Paraburkholderia bacteria are contacted at a rate of about 5×10¹³ bacteria per hectare.

16. A method for preventing and/or controlling pests on a plant, and/or on a temporary part of a plant and/or being located inside of a plant comprising contacting Paraburkholderia bacteria to said plant and/or to said temporary part of said plant and/or a locus for plant growth.

17. The method of claim 16, wherein said Paraburkholderia bacteria comprises bacteria of the species Paraburkholderia phytofirmans, preferably wherein said Paraburkholderia phytofirmans comprises bacteria of the strain Paraburkholderia phytofirmans PsJN.

18. The method of claims 16 and 17, wherein said preventing and/or controlling of pests increases undamaged fruit productivity of said plant.

19. The method of any one of claims 16 to 18, wherein said pests comprise Erwinia amylovora.

20. The method of any one of claims 16 to 19, wherein said plant belongs to the Rosaceae family of plants.

21. The method of claim 20, wherein said Rosaceae family of plants comprises pears, peaches, apples, quinces, apricots, plums, cherries, raspberries, loquats, strawberries, rose hips, hawthorns, almonds and roses, preferably wherein said Rosaceae family of plants comprises pears, peaches and apples.

22. The method of any one of claims 16 to 21, wherein said temporary part of said plant is selected from the group consisting of flowers, leaves and fruits.

23. The method of any one of claims 16 to 22, wherein said Paraburkholderia bacteria are formulated in a composition, preferably wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.

24. The method of claim 23, wherein said composition further comprises at least a second agent and optionally secreted metabolites of said Paraburkholderia bacteria.

25. The method of claim 24, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores preferably wherein said at least second agent is a surfactant.

26. The method of claim 25, wherein said surfactant is a polyethersiloxane/polyether trisiloxane.

27. The method of claims 25 and 26, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.

28. The method of any one of claims 16 to 27, wherein said Paraburkholderia bacteria or said composition comprising said Paraburkholderia bacteria is contacted to said plant and/or said temporary part of said plant and/or said locus for plant growth, preferably wherein said Paraburkholderia bacteria or said composition comprising said Paraburkholderia bacteria is contacted to the flowers of said plant.

29. The method of claim 28, wherein said contacting is performed at least once, at least twice, at least three times, at least four times or at least five times.

30. The method of claims 28 and 29, wherein Paraburkholderia bacteria are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein Paraburkholderia bacteria are contacted at a rate of about 5×10¹³ bacteria per hectare.

31. The method of any one of claims 16 to 30, wherein said locus for plant growth is soil.

32. A composition for preventing and/or controlling pests on a plant and/or on a temporary part of a plant and/or being located inside of a plant comprising Paraburkholderia bacteria.

33. The composition of claim 32, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.

34. The composition of claims 32 and 33, wherein said preventing and/or controlling of pests increases undamaged fruit productivity of said plant.

35. The composition of any one of claims 32 to 34, wherein said pests comprise Erwinia amylovora.

36. The composition of any one of claims 32 to 35, wherein said plant belongs to the Rosaceae family of plants.

37. The composition of claim 36, wherein said Rosaceae family of plants comprises pears, peaches, apples, quinces, apricots, plums, cherries, raspberries, loquats, strawberries, rose hips, hawthorns, almonds and roses, preferably wherein said Rosaceae family of plants comprises pears, peaches and apples.

38. The composition of any one of claims 32 to 37, wherein said temporary part of said plant is selected from the group consisting of flowers, leaves and fruits.

39. The composition of any one of claims 32 to 38, wherein said composition is formulated as a bacterial suspension with at least 10⁴-10¹¹ bacteria/ml, preferably wherein said composition is formulated as a bacterial suspension with at least 10⁶-10⁸ bacteria/ml and most preferably wherein said composition is formulated as a bacterial suspension of about 10⁸ bacteria/ml.

40. The composition of any one of claims 32 to 39, wherein said composition further comprises at least a second agent and optionally secreted metabolites of said Paraburkholderia bacteria.

41. The composition of claim 40, wherein said at least second agent is selected from the group consisting of inorganic, organic, ionic, non-ionic compounds, sugars, amino acids, chemical and/or organic fertilizers, fungicides, nematicides, insecticides and/or defensive compounds against herbivores preferably wherein said at least second agent is a surfactant.

42. The composition of claim 41, wherein said surfactant is a polyethersiloxane/polyether trisiloxane.

43. The composition of claims 41 and 42, wherein said surfactant constitutes about 0.01, about 0.02, about 0.03, about 0.04, about 0.05, about 0.06, about 0.07, about 0.08, about 0.09, about 0.1, about 0.15, about 0.2, about 0.25, about 0.3, about 0.5 volume percent of said composition comprising said Paraburkholderia bacteria, preferably wherein said surfactant constitutes about 0.1-0.2 volume percent of said composition comprising said Paraburkholderia bacteria.

44. The composition of any one of claims 32 to 43, wherein said composition is contacted to said plant and/or said temporary part of said plant, preferably wherein said composition is contacted to the flowers of said plant.

45. The composition of claim 44, wherein said contacting is performed at least once, at least twice, at least three times, at least four times or at least five times.

46. The composition of claims 44 and 45, wherein Paraburkholderia bacteria being contained in said composition are contacted at a rate of at least 10⁸, 10⁹, 10¹⁰, 10¹¹, 10¹², 10¹³, 10¹⁴, 10¹⁵, 10¹⁶, 10¹⁷ bacteria per hectare, preferably, wherein Paraburkholderia bacteria are contacted at a rate of about 5×10¹³ bacteria per hectare.

47. The composition of any one of claims 32 to 46, wherein said composition is a formulated product, preferably wherein said formulated product is a ready-to-use product or a product that is to be diluted with a suitable diluent prior to use.

48. A kit comprising the composition of claim 47, preferably together with instructions regarding the use of the method of any one of claims 16 to 31.

49. Use of Paraburkholderia bacteria for the preparation of a composition, wherein said composition can be a dried powder or a spraying agent.

50. The use of Paraburkholderia bacteria according to claim 49, wherein said Paraburkholderia bacteria comprise bacteria of the species Paraburkholderia phytofirmans, preferably wherein said Paraburkholderia phytofirmans comprises bacteria of the strain Paraburkholderia phytofirmans PsJN.
